SEQUENCE LISTING (40 sequences)

SEQ ID NO: 1 - RNA, 50 nt, Homo sapiens
  source 1..50: mol_type = other RNA
tggtgggcggtgttgtcatagcgacagtgatcgtcatcaccttggtgatg

SEQ ID NO: 2 - DNA, 38 bp, synthetic construct
  misc_feature 1..24: note = RNA
  source 1..38: mol_type = other DNA
  misc_feature 25..26: note = DNA
  misc_feature 27..38: note = RNA
  modified_base 24: mod_base = OTHER; note = 2-Prime-2-prime-difluoro substituted Cytidine
  modified_base order(7..11,13,14,20,21,23,29): mod_base = OTHER; note = 2-Prime-O-methoxyethyl modified nucleosides
  modified_base order(1..7,15..20,25..27,31..38): mod_base = OTHER; note = Phosphorothioate linkage
  modified_base order(1..6,15..19,27,28,30..38): mod_base = OTHER; note = 2-Prime-O-Methyl modified nucleosides
  modified_base order(12,22): mod_base = OTHER; note = 2-Prime-O-methoxyethyl modified 5-methyl-Cytidine
caaggtgatgacgatcactgtcgccatgacaacaccgc

SEQ ID NO: 3 - DNA, 38 bp, synthetic construct; identical to SEQ ID NO: 2
  misc_feature 1..25: note = RNA
  source 1..38: mol_type = other DNA
  misc_feature 26: note = DNA
  misc_feature 27..38: note = RNA
  modified_base order(1..7,15..20,25..27,31..38): mod_base = OTHER; note = Phosphorothioate linkage
  modified_base order(7..11,13,14,20,21,23,29): mod_base = OTHER; note = 2-Prime-O-methoxyethyl modified nucleosides
  modified_base order(1..6,15..19,27,28,30..38): mod_base = OTHER; note = 2-Prime-O-Methyl modified nucleosides
  modified_base 25: mod_base = OTHER; note = 2-Prime-2-prime-difluoro substituted Cytidine
  modified_base order(12,22,24): mod_base = OTHER; note = 2-Prime-O-methoxyethyl modified 5-methyl-Cytidine

SEQ ID NO: 4 - DNA, 38 bp, synthetic construct; identical to SEQ ID NO: 2
  misc_feature 1..24: note = RNA
  source 1..38: mol_type = other DNA
  misc_feature 25..26: note = DNA
  misc_feature 27..38: note = RNA
  modified_base order(1..7,15..20,25..27,31..38): mod_base = OTHER; note = Phosphorothioate linkage
  modified_base 30: mod_base = OTHER; note = 2-Prime-2-prime-difluoro substituted Cytidine
  modified_base order(7..11,13,14,20,21,23,29): mod_base = OTHER; note = 2-Prime-O-methoxyethyl modified nucleosides
  modified_base order(12,22,24): mod_base = OTHER; note = 2-Prime-O-methoxyethyl modified 5-methyl-Cytidine
  modified_base order(1..6,15..19,27,28,31..38): mod_base = OTHER; note = 2-Prime-O-Methyl modified nucleosides

SEQ ID NO: 5 - DNA, 38 bp, synthetic construct; identical to SEQ ID NO: 2
  misc_feature 1..24: note = RNA
  source 1..38: mol_type = other DNA
  misc_feature 25..26: note = DNA
  misc_feature 27..38: note = RNA
  modified_base order(1..7,15..20,25..27,31..38): mod_base = OTHER; note = Phosphorothioate linkage
  modified_base order(22,24): mod_base = OTHER; note = 2-Prime-2-prime-difluoro substituted Cytidine
  modified_base order(7..11,13,14,20,21,23,29): mod_base = OTHER; note = 2-Prime-O-methoxyethyl modified nucleosides
  modified_base 12: mod_base = OTHER; note = 2-Prime-O-methoxyethyl modified 5-methyl-Cytidine
  modified_base order(1..6,15..19,27,28,30..38): mod_base = OTHER; note = 2-Prime-O-Methyl modified nucleosides

SEQ ID NO: 6 - DNA, 38 bp, synthetic construct; identical to SEQ ID NO: 2
  misc_feature 1..24: note = RNA
  source 1..38: mol_type = other DNA
  misc_feature 25..26: note = DNA
  misc_feature 27..38: note = RNA
  modified_base order(1..7,15..20,25..27,31..38): mod_base = OTHER; note = Phosphorothioate linkage
  modified_base order(7..11,13,14,20,21,23,29): mod_base = OTHER; note = 2-Prime-O-methoxyethyl modified nucleosides
  modified_base order(12,22,24): mod_base = OTHER; note = 2-Prime-O-methoxyethyl modified 5-methyl-Cytidine
  modified_base order(1..6,15..19,27,28,30..38): mod_base = OTHER; note = 2-Prime-O-Methyl modified nucleosides

SEQ ID NO: 7 - DNA, 38 bp, synthetic construct
  misc_feature 24..26: note = DNA
  source 1..38: mol_type = other DNA
  misc_feature 1..23: note = RNA
  misc_feature 27..38: note = RNA
  modified_base order(1..7,15..20,31..38): mod_base = OTHER; note = Phosphorothioate linkage
  modified_base order(1..23,27..38): mod_base = OTHER; note = 2-Prime-O-Methyl modified nucleosides
gcattgaaagccactcctccaggtcacagctggcagaa

SEQ ID NO: 8 - RNA, 25 nt, synthetic construct
  source 1..25: mol_type = other RNA
  modified_base order(1..10,14,17..25): mod_base = OTHER; note = 2-Prime-O-Methyl modified nucleosides
  modified_base 11: mod_base = OTHER; note = 2-Prime-O-methoxyethyl modified 5-methyl-Cytidine
  modified_base 16: mod_base = OTHER; note = 2-Prime-O-methoxyethyl modified nucleosides
  modified_base 15: mod_base = OTHER; note = 2- Prime-fluoro nucleosides
  modified_base 13^14: mod_base = OTHER; note = Methylphosphonate linkage
  modified_base order(1..13,14..25): mod_base = OTHER; note = Phosphorothioate linkage
atcactgtcgccatgacaacaccgc

SEQ ID NO: 9 - RNA, 25 nt, synthetic construct
  source 1..25: mol_type = other RNA
  modified_base order(1..13,14..25): mod_base = OTHER; note = Phosphorothioate linkage
  modified_base 13^14: mod_base = OTHER; note = Methylphosphonate linkage
  modified_base order(1..10,14,17..25): mod_base = OTHER; note = 2-Prime-O-Methyl modified nucleosides
  modified_base 12: mod_base = OTHER; note = 2-Prime-2-prime-difluoro substituted Cytidine
  modified_base 16: mod_base = OTHER; note = 2-Prime-O-methoxyethyl modified nucleosides
  modified_base 11: mod_base = OTHER; note = 2-Prime-O-methoxyethyl modified 5-methyl-Cytidine
  modified_base 15: mod_base = OTHER; note = 2- Prime-fluoro nucleosides
atcactgtcgccatgacaacaccgc

SEQ ID NO: 10 - RNA, 25 nt, synthetic construct
  source 1..25: mol_type = other RNA
  modified_base order(1..13,14..25): mod_base = OTHER; note = Phosphorothioate linkage
  modified_base 13^14: mod_base = OTHER; note = Methylphosphonate linkage
  modified_base order(1..10,14,17..25): mod_base = OTHER; note = 2-Prime-O-Methyl modified nucleosides
  modified_base 11..12: mod_base = OTHER; note = 2-Prime-2-prime-difluoro substituted Cytidine
  modified_base 16: mod_base = OTHER; note = 2-Prime-O-methoxyethyl modified nucleosides
  modified_base 15: mod_base = OTHER; note = 2- Prime-fluoro nucleosides
atcactgtcgccatgacaacaccgc

SEQ ID NO: 11 - RNA, 25 nt, synthetic construct
  source 1..25: mol_type = other RNA
  modified_base order(1..13,14..25): mod_base = OTHER; note = Phosphorothioate linkage
  modified_base 13^14: mod_base = OTHER; note = Methylphosphonate linkage
  modified_base order(1..8,10,14,17..25): mod_base = OTHER; note = 2-Prime-O-Methyl modified nucleosides
  modified_base order(9,11,12): mod_base = OTHER; note = 2-Prime-2-prime-difluoro substituted Cytidine
  modified_base 15: mod_base = OTHER; note = 2- Prime-fluoro nucleoside
  modified_base 16: mod_base = OTHER; note = 2-Prime-O-methoxyethyl modified nucleoside
atcactgtcgccatgacaacaccgc

SEQ ID NO: 12 - RNA, 50 nt, Mus musculus
  source 1..50: mol_type = other RNA
tggtgggcggcgttgtcatagcaaccgtgattgtcatcaccctggtgatg

SEQ ID NO: 13 - DNA, 25 bp, synthetic construct
  misc_feature 12..13: note = DNA
  source 1..25: mol_type = other DNA
  misc_feature 1..11: note = RNA
  misc_feature 14..25: note = RNA
  modified_base order(1..2,24..25): mod_base = OTHER; note = (1,3-dimethylimidazolidin-2-ylidene) phosphoramidate linkage
  modified_base 13^14: mod_base = OTHER; note = Methylphosphonate linkage
  modified_base order(2..13,14..24): mod_base = OTHER; note = Phosphorothioate linkage
  modified_base order(6,8,10,15,17,19,21): mod_base = OTHER; note = 2- Prime-fluoro nucleosides
  modified_base 16: mod_base = OTHER; note = 2-Prime-O-methoxyethyl modified nucleoside
  modified_base 11: mod_base = OTHER; note = 2-Prime-O-methoxyethyl modified 5-methyl-Cytidine
  modified_base order(1..5,7,9,14,18,20,22..25): mod_base = OTHER; note = 2-Prime-O-Methyl modified nucleosides
atcacggttgccatgacaacgccgc

SEQ ID NO: 14 - DNA, 25 bp, synthetic construct
  misc_feature 13: note = DNA
  source 1..25: mol_type = other DNA
  misc_feature 1..12: note = RNA
  misc_feature 14..25: note = RNA
  modified_base order(1..2,24..25): mod_base = OTHER; note = (1,3-dimethylimidazolidin-2-ylidene) phosphoramidate linkage
  modified_base 13^14: mod_base = OTHER; note = Methylphosphonate linkage
  modified_base order(2..13,14..24): mod_base = OTHER; note = Phosphorothioate linkage
  modified_base order(6,8,10,15,17,19,21): mod_base = OTHER; note = 2- Prime-fluoro nucleosides
  modified_base 16: mod_base = OTHER; note = 2-Prime-O-methoxyethyl modified nucleoside
  modified_base 11: mod_base = OTHER; note = 2-Prime-O-methoxyethyl modified 5-methyl-Cytidine
  modified_base 12: mod_base = OTHER; note = 2-Prime-2-prime-difluoro substituted Cytidine
  modified_base order(1..5,7,9,14,18,20,22..25): mod_base = OTHER; note = 2-Prime-O-Methyl modified nucleosides
atcacggttgccatgacaacgccgc

SEQ ID NO: 15 - RNA, 64 nt, Homo sapiens
  source 1..64: mol_type = mRNA
gaggactcctctgtctttgcccagagcatcccgtggaacctggagcggattacccctccacggt

SEQ ID NO: 16 - DNA, 27 bp, synthetic construct
  misc_feature 14..15: note = DNA
  source 1..27: mol_type = other DNA
  misc_feature 1..13: note = RNA
  misc_feature 16..27: note = RNA
  modified_base 15: mod_base = OTHER; note = Deoxyinosine
  modified_base 14: mod_base = OTHER; note = DNA nucleotide carrying a Benner's base
  modified_base order(1..2,26..27): mod_base = OTHER; note = (1,3-dimethylimidazolidin-2-ylidene) phosphoramidate linkage
  modified_base 15^16: mod_base = OTHER; note = Methylphosphonate linkage
  modified_base order(4..13,14..15,17..26): mod_base = OTHER; note = Phosphorothioate linkage
  modified_base order(8,10,12,17,19,21,23): mod_base = OTHER; note = 2- Prime-fluoro nucleosides
  modified_base order(1,2,13,18): mod_base = OTHER; note = 2-Prime-O-methoxyethyl 5-methyl-Cytidine
  modified_base order(4..7,9,11,16,20,22,24..27): mod_base = OTHER; note = 2-Prime-O-Methyl modified nucleosides
  modified_base 3: mod_base = OTHER; note = 2-Prime-O-methoxyethyl modified nucleoside
ccacgggatgctcnnggcaaagacaga

SEQ ID NO: 17 - DNA, 27 bp, synthetic construct
  misc_feature 1..14: note = RNA
  source 1..27: mol_type = other DNA
  misc_feature 15: note = DNA
  misc_feature 16..27: note = RNA
  modified_base order(1..2,26..27): mod_base = OTHER; note = (1,3-dimethylimidazolidin-2-ylidene) phosphoramidate linkage
  modified_base order(4..13,14..15,17..26): mod_base = OTHER; note = Phosphorothioate linkage
  modified_base 15^16: mod_base = OTHER; note = Methylphosphonate linkage
  modified_base 15: mod_base = OTHER; note = Deoxyinosine
  modified_base order(1,2,13,18): mod_base = OTHER; note = 2-Prime-O-methoxyethyl 5-methyl-Cytidine
  modified_base order(4..7,9,11,16,20,22,24..27): mod_base = OTHER; note = 2-Prime-O-Methyl modified nucleosides
  modified_base 14: mod_base = OTHER; note = 2-prime-2-prime-difluoro substituted Cytidine
  modified_base order(8,10,12,17,19,21,23): mod_base = OTHER; note = 2- Prime-fluoro nucleosides
  modified_base 3: mod_base = OTHER; note = 2-Prime-O-methoxyethyl modified nucleoside
ccacgggatgctccnggcaaagacaga

SEQ ID NO: 18 - DNA, 27 bp, synthetic construct
  misc_feature 14..15: note = DNA
  source 1..27: mol_type = other DNA
  misc_feature 1..13: note = RNA
  misc_feature 16..27: note = RNA
  modified_base order(1,2,13,18): mod_base = OTHER; note = 2-Prime-O-methoxyethyl 5-methyl-Cytidine
  modified_base order(4..7,9,11,16,20,22,24..27): mod_base = OTHER; note = 2-Prime-O-Methyl modified nucleosides
  modified_base order(8,10,12,17,19,21,23): mod_base = OTHER; note = 2- Prime-fluoro nucleosides
  modified_base 14: mod_base = OTHER; note = DNA nucleotide carrying a Benner's base
  modified_base 15: mod_base = OTHER; note = Deoxyinosine
  modified_base order(1..2,26..27): mod_base = OTHER; note = (1,3-dimethylimidazolidin-2-ylidene) phosphoramidate linkage
  modified_base 15^16: mod_base = OTHER; note = Methylphosphonate linkage
  modified_base order(4..13,14..15,17..26): mod_base = OTHER; note = Phosphorothioate linkage
  modified_base 3: mod_base = OTHER; note = 2-Prime-O-methoxyethyl modified nucleoside
ccatgggatgctcnnggcgaagacaga

SEQ ID NO: 19 - DNA, 27 bp, synthetic construct
  misc_feature 15: note = DNA
  source 1..27: mol_type = other DNA
  misc_feature 1..14: note = RNA
  misc_feature 16..27: note = RNA
  modified_base order(1..2,26..27): mod_base = OTHER; note = (1,3-dimethylimidazolidin-2-ylidene) phosphoramidate linkage
  modified_base 15^16: mod_base = OTHER; note = Methylphosphonate linkage
  modified_base 15: mod_base = OTHER; note = Deoxyinosine
  modified_base order(4..13,14..15,17..26): mod_base = OTHER; note = Phosphorothioate linkage
  modified_base order(1,2,13,18): mod_base = OTHER; note = 2-Prime-O-methoxyethyl 5-methyl-Cytidine
  modified_base order(8,10,12,17,19,21,23): mod_base = OTHER; note = 2- Prime-fluoro nucleosides
  modified_base 14: mod_base = OTHER; note = 2-Prime-2-prime-difluoro substituted Cytidine
  modified_base order(4..7,9,11,16,20,22,24..27): mod_base = OTHER; note = 2-Prime-O-Methyl modified nucleosides
  modified_base 3: mod_base = OTHER; note = 2-Prime-O-methoxyethyl modified nucleoside
ccatgggatgctccnggcgaagacaga

SEQ ID NO: 20 - DNA, 20 bp, synthetic construct
  source 1..20: mol_type = other DNA
ctcgacgcaagccataacac

SEQ ID NO: 21 - DNA, 20 bp, synthetic construct
  source 1..20: mol_type = other DNA
tggaccgactggaaacgtag

SEQ ID NO: 22 - DNA, 21 bp, synthetic construct
  source 1..21: mol_type = other DNA
tgggttgacaaatatcaagac

SEQ ID NO: 23 - DNA, 24 bp, synthetic construct
  source 1..24: mol_type = other DNA
caccatgatgaatggatgtgtact

SEQ ID NO: 24 - DNA, 17 bp, synthetic construct
  source 1..17: mol_type = other DNA
gcaatcattggactcat

SEQ ID NO: 25 - DNA, 36 bp, synthetic construct
  source 1..36: mol_type = other DNA
ggtgggcggtgttgtcatrgcgacagtgatcgtcat

SEQ ID NO: 26 - DNA, 26 bp, synthetic construct
  source 1..26: mol_type = other DNA
tgtgcgtgtgtcactagcgagcagtg

SEQ ID NO: 27 - DNA, 17 bp, synthetic construct
  source 1..17: mol_type = other DNA
cattggactcatggtgg

SEQ ID NO: 28 - DNA, 16 bp, synthetic construct
  source 1..16: mol_type = other DNA
cagcatcaccaaggtg

SEQ ID NO: 29 - DNA, 18 bp, synthetic construct
  source 1..18: mol_type = other DNA
  modified_base order(5,10..12): mod_base = OTHER; note = Locked Nucleic Acid nucleotides
tgttgtcatagcgacagt

SEQ ID NO: 30 - DNA, 18 bp, synthetic construct
  source 1..18: mol_type = other DNA
  modified_base 10..11: mod_base = OTHER; note = Locked Nucleic Acid nucleotides
tgttgtcatggcgacagt

SEQ ID NO: 31 - DNA, 23 bp, synthetic construct
  source 1..23: mol_type = other DNA
tgctggagctggccttgaagttg

SEQ ID NO: 32 - DNA, 22 bp, synthetic construct
  source 1..22: mol_type = other DNA
ctggtattcatccgcccggtac

SEQ ID NO: 33 - DNA, 16 bp, synthetic construct
  source 1..16: mol_type = other DNA
ttccgaggaggacggc

SEQ ID NO: 34 - DNA, 22 bp, synthetic construct
  source 1..22: mol_type = other DNA
gtctaggagatacacctccacc

SEQ ID NO: 35 - DNA, 21 bp, synthetic construct
  source 1..21: mol_type = other DNA
agtttctccaggagtgggaag

SEQ ID NO: 36 - DNA, 20 bp, synthetic construct
  source 1..20: mol_type = other DNA
agcacctggcaatggcgtag

SEQ ID NO: 37 - DNA, 17 bp, synthetic construct
  source 1..17: mol_type = other DNA
  modified_base 8..10: mod_base = OTHER; note = Locked Nucleic Acid nucleotides
ctttgcccagagcatcc

SEQ ID NO: 38 - DNA, 17 bp, synthetic construct
  source 1..17: mol_type = other DNA
  modified_base 8..10: mod_base = OTHER; note = Locked Nucleic Acid nucleotides
ctttgcccggagcatcc

SEQ ID NO: 39 - DNA, 18 bp, synthetic construct
  source 1..18: mol_type = other DNA
agcgcatggaggcccaag

SEQ ID NO: 40 - DNA, 20 bp, synthetic construct
  source 1..20: mol_type = other DNA
ctgctggagctgacggaggc

(19) United States
(12) Patent Application Publication
(10) Pub. No.: US 2026/0027237 A1
(43) Pub. Date: Jan. 29, 2026

(54) CHEMICALLY MODIFIED OLIGONUCLEOTIDES FOR ADAR-MEDIATED RNA EDITING

(71) Applicant: ProQR Therapeutics II B.V., Leiden (NL)

(72) Inventor: Peter Christian DE VISSER, Leiden (NL)

(73) Assignee: ProQR Therapeutics II B.V., Leiden (NL)

(21) Appl. No.: 18/994,574

(22) PCT Filed: Jul. 14, 2023
(86) PCT No.: PCT/EP2023/069609

§ 371 Date: Jan. 14, 2025

Related U.S. Application Data
(60) Provisional application No. 63/389,441, filed on Jul. 15, 2022.

(30) Foreign Application Priority Data
Sep. 30, 2022 (GB) 2214347.3

(51) Int. Cl. A61K 48/00; C12N 9/78; C12N 15/11

(52) U.S. Cl. CPC A61K 48/0058; C12N 9/78; C12N 15/11; C12Y 305/04004; C12N 2310/20; C12N 2310/322

(57) ABSTRACT

The invention relates to antisense oligonucleotides that can form a double stranded nucleic acid complex with a target RNA molecule, wherein the double stranded nucleic acid complex is capable of recruiting an adenosine deaminating enzyme for deamination of a target adenosine in the target RNA molecule, wherein the nucleotide in the AON that is directly opposite the target adenosine comprises a 2′,2′-disubstitution, preferably a 2′,2′-difluro substitution in the ribose moiety.

### TECHNICAL FIELD

The invention relates to the field of medicine, and more specifically to the field of RNA editing wherein an RNA molecule, such as a pre-mRNA or a mRNA molecule in a cell is targeted by an antisense oligonucleotide (AON) to change the chemical properties of a specific nucleotide within the target RNA molecule. The invention relates to RNA-editing AONs (also herein and elsewhere referred to as ‘EONs’) that comprise chemical modifications at specified positions to improve their in vivo and in vitro RNA editing effect.

### BACKGROUND

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are adenosine (A) to inosine (I) conversions and cytidine (C) to uridine (U) conversions, which occur through enzymes called Adenosine Deaminases acting on RNA (ADAR) and APOBEC/AID (cytidine deaminases that act on RNA), respectively.

ADAR is a multi-domain protein, comprising a catalytic domain, and two to three double-stranded RNA recognition domains, depending on the enzyme in question. Each recognition domain recognizes a specific double stranded RNA (dsRNA) sequence and/or conformation. The catalytic domain does also play a role in recognizing and binding a part of the dsRNA helix, although the key function of the catalytic domain is to convert an A into I in a nearby, predefined, position in the target RNA, by deamination of the nucleobase. Inosine is read as guanosine by the translational machinery of the cell, meaning that, if an edited adenosine is in a coding region of an mRNA or pre-mRNA, it can recode the protein sequence. A to I conversions may also occur in 5′ non-coding sequences of a target mRNA, creating new translational start sites upstream of the original start site, which gives rise to N-terminally extended proteins, or in the 3′ UTR or other non-coding parts of the transcript, which may affect the processing and/or stability of the RNA. In addition, A to I conversions may take place in splice elements in introns or exons in pre-mRNAs, thereby altering the pattern of splicing. As a result, exons may be included or skipped. The enzymes catalysing adenosine deamination are within an enzyme family of ADARs, which include human deaminases hADAR1 and hADAR2, as well as hADAR3. However, for hADAR3 no deaminase activity has been demonstrated.

The use of oligonucleotides to edit a target RNA applying adenosine deaminase has been described (e.g., Woolf et al. 1995. PNAS 92:8298-8302; Montiel-Gonzalez et al. PNAS 2013, 110 (45): 18285-18290; Vogel et al. 2014. Angewandte Chemie Int Ed 53:267-271). A disadvantage of the method described by Montiel-Gonzalez et al. (2013) is the need for a fusion protein consisting of the boxB recognition domain of bacteriophage lambda N-protein, genetically fused to the adenosine deaminase domain of a truncated natural ADAR protein. It requires target cells to be either transduced with the fusion protein, which is a major hurdle, or that target cells are transfected with a nucleic acid construct encoding the engineered adenosine deaminase fusion protein for expression. The system described by Vogel et al. (2014) suffers from similar drawbacks, in that it is not clear how to apply the system without having to genetically modify the ADAR first and subsequently transfect or transform the cells harboring the target RNA, to provide the cells with this genetically engineered protein. U.S. Pat. No. 9,650,627 describes a similar system. The oligonucleotides of Woolf et al. (1995) that were 100% complementary to the target RNA sequences suffered from severe lack of specificity: nearly all adenosines in the target RNA strand that was complementary to the antisense oligonucleotide were edited.

It is known that ADAR may act on any dsRNA. Through a process sometimes referred to as ‘promiscuous editing’, the enzyme will edit multiple A's in the dsRNA. Hence, there is a need for methods and means that circumvent such promiscuous editing and only target specific adenosines in a target RNA molecule to become therapeutic applicable. Vogel et al. (2014) showed that such off-target editing can be suppressed by using 2′-O-Me-modified nucleosides in the oligonucleotide at positions opposite to adenosines that should not be edited and used a non-modified nucleoside directly opposite to the specifically targeted adenosine on the target RNA. However, the specific editing effect at the target nucleotide has not been shown to take place without the use of recombinant ADAR enzymes having covalent bonds with the AON. Several publications have now shown that the recruitment of endogenous ADAR (hence without the need for an exogenous and/or recombinant source) is feasible while maintaining a specificity in which a single adenosine within a target RNA molecule can be targeted and deaminated to an inosine. WO2016/097212 discloses antisense oligonucleotides (AONs) for the targeted editing of RNA, wherein the AONs are characterized by a sequence that is complementary to a target RNA sequence (therein referred to as the ‘targeting portion’) and by the presence of a stem-loop/hairpin structure (therein referred to as the ‘recruitment portion’), which is preferably non-complementary to the target RNA. Such oligonucleotides are referred to as ‘self-looping AONs’. The recruitment portion acts in recruiting a natural ADAR enzyme present in the cell to the dsRNA formed by hybridization of the target sequence with the targeting portion. Due to the recruitment portion, there is no need for conjugated entities or presence of modified recombinant ADAR enzymes. WO2016/097212 describes the recruitment portion as being a stem-loop structure mimicking either a natural substrate (e.g., the GluB receptor) or a Z-DNA structure known to be recognized by the dsRNA binding domains, or Z-DNA binding domains, of ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion as described is an intramolecular stem-loop structure, formed within the AON itself, and are thought to attract (endogenous) ADAR. Similar stem-loop structure-comprising systems for RNA editing have been described in WO2017/050306, WO2020/001793, WO2017/010556, WO2020/246560, and WO2022/078995.

WO2017/220751 and WO2018/041973 describe a next generation type of AONs that do not comprise such a stem-loop structure but that are (almost fully) complementary to the targeted area except for one or more mismatching nucleotides, wobbles, or bulges. The sole mismatch may be at the site of the nucleoside opposite the target adenosine, but in other embodiments AONs were described with multiple bulges and/or wobbles when attached to the target sequence area. It appeared possible to achieve in vitro, ex vivo and in vivo RNA editing with AONs lacking a stem-loop structure and with endogenous ADAR enzymes when the sequence of the AON was carefully selected such that it could attract/recruit ADAR. The ‘orphan nucleoside’, which is defined as the nucleoside in the AON that is positioned directly opposite the target adenosine in the target RNA molecule, did not carry a 2′-O-Me modification. The orphan nucleoside could also be a deoxyribonucleoside (DNA, carrying no 2′ modification in the sugar entity), wherein the remainder of the AON did carry 2′-O-alkyl modifications at the sugar entity (such as 2′-O-Me), or the nucleotides directly surrounding the orphan nucleoside contained particular chemical modifications (such as DNA in comparison to RNA) that further improved the RNA editing efficiency and/or increased the resistance against nucleases. Such effects could even be further improved by using sense oligonucleotides (SONs) that ‘protected’ the AONs against breakdown (described in WO2018/134301). The use of chemical modifications and particular structures in oligonucleotides that could be used in ADAR-mediated editing of specific adenosines in a target RNA have been the subject of numerous publications in the field, such as WO2019/111957, WO2019/158475, WO2020/165077, WO2020/201406, WO2020/211780, WO2021/008447, WO2021/020550, WO2021/060527, WO2021/117729, WO2021/136408, WO2021/182474, WO2021/216853, WO2021/242778, WO2021/242870, WO2021/242889, WO2022/007803, WO2022/018207, WO2022/026928, and WO2022/124345. The use of specific sugar moieties has been disclosed in for instance WO2020/154342, WO2020/154343, WO2020/154344, WO2022/103839, and WO2022/103852, whereas the use of stereo-defined linker moieties (in general for oligonucleotides that for instance can be used for exon skipping, in gapmers, in siRNA, or specifically for RNA-editing oligonucleotides, related to a wide variety of target sequences) has been described in WO2011/005761, WO2014/010250, WO2014/012081, WO2015/107425, WO2017/015575 (HTT), WO2017/062862, WO2017/160741, WO2017/192664, WO2017/192679 (DMD), WO2017/198775, WO2017/210647, WO2018/067973, WO2018/098264, WO2018/223056 (PNPLA3), WO2018/223073 (APOC3), WO2018/223081 (PNPLA3), WO2018/237194, WO2019/032607 (C9orf72), WO2019/055951, WO2019/075357 (SMA/ALS), WO2019/200185 (DM1), WO2019/217784 (DM1), WO2019/219581, WO2020/118246 (DM1), WO2020/160336 (HTT), WO2020/191252, WO2020/196662, WO2020/219981 (USH2A), WO2020/219983 (RHO), WO2020/227691 (C9orf72), WO2021/071788 (C9orf72), WO2021/071858, WO2021/178237 (MAPT), WO2021/234459, WO2021/237223, and WO2022/099159. Next to these disclosures, an extensive number of publications relate to the targeting of specific RNA target molecules, or specific adenosines within such RNA target molecules, be it to repair a mutation that resulted in a premature stop codon, or other mutation causing disease. Examples of such disclosures in which adenosines are targeted within specified target RNA molecules are WO2020/157008 and WO2021/136404 (USH2A); WO2021/113270 (APP); WO2021/113390 (CMT1A); WO2021/209010 (IDUA, Hurler syndrome); WO2021/231673 and WO2021/242903 (LRRK2); WO2021/231675 (ASS1); WO2021/231679 (GJB2); WO2019/071274 and WO2021/231680 (MECP2); WO2021/231685 and WO2021/231692 (OTOF, autosomal recessive non-syndromic hearing loss); WO2021/231691 (XLRS); WO2021/231698 (argininosuccinate lyase deficiency); WO2021/130313 and WO2021/231830 (ABCA4); and WO2021/243023 (SERPINA1).

Despite the numerous and wide variety of achievements outlined above, there remains a need for improved compounds that can utilise (endogenous) cellular pathways and enzymes that have deaminase activity, such as naturally expressed ADAR enzymes to edit specifically and more efficiently endogenous nucleic acids in mammalian cells, even in whole organisms, to alleviate disease.

### SUMMARY OF THE INVENTION

The invention relates to an antisense oligonucleotide (AON) that can form a double stranded nucleic acid complex with a target RNA molecule, wherein the double stranded nucleic acid complex is capable of recruiting an adenosine deaminating enzyme for deamination of a target adenosine in the target RNA molecule, wherein the nucleotide in the AON that is opposite the target adenosine is the orphan nucleotide, and wherein the orphan nucleotide has the structure of formula I:

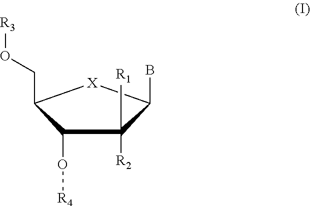

wherein: X is O, NH, CH₂, Se, or S; B is a nitrogenous base selected from the group consisting of: cytosine, uracil, isouracil, N3-glycosylated uracil, pseudoisocytosine, 8-oxo-adenine, and 6-amino-5-nitro-2(1H)-pyridone; R₁ and R₂ are both selected, independently, from OH, F or CH₃; R₃ is the part of the AON that is 5′ of the orphan nucleotide, consisting of 7 to 30 nucleotides; and R₄ is the part of the AON that is 3′ of the orphan nucleotide, consisting of 4 to 25 nucleotides. In one embodiment R₁ or R₂ is F, and R₁ or R₂ is either CH₃ or also F. In another embodiment R₁ and R₂ are both F.

The invention further relates to a pharmaceutical composition comprising an AON according to the invention, and a pharmaceutically acceptable carrier or diluent. The invention also relates to an AON according to the invention, or a pharmaceutical composition according to the invention, for use in the treatment, amelioration, slowing down progression, or prevention of a genetic disease or a disease that is treatable using an AON of the present invention.

The invention also relates to a method for the deamination of at least one target adenosine present in a target RNA molecule in a cell, the method comprising the steps of: (i) providing the cell with an AON according to the invention, or a pharmaceutical composition according to the invention; (ii) allowing annealing of the AON to the target RNA molecule to form a double stranded nucleic acid complex capable of recruiting an adenosine deaminating enzyme in the cell, preferably an endogenous adenosine deaminating enzyme, more preferably ADAR2; (iii) allowing the adenosine deaminating enzyme to deaminate the target adenosine in the target RNA molecule; and (iv) optionally identifying the presence of the deaminated adenosine in the target RNA molecule. In a further preferred embodiment, step (iv) comprises: (a) sequencing a region of the target RNA molecule, wherein the region comprises the deaminated target adenosine; (b) assessing the presence of a functional, elongated, full length and/or wild type protein when the target adenosine has resulted in a stop codon; or (c) using a functional read-out, wherein the target RNA molecule after the deamination encodes a functional, full length, elongated and/or wild type protein.

### BRIEF DESCRIPTION OF THE DRAWINGS

One or more embodiments of the invention will now be described, by way of example only, with reference to the accompanying drawings, in which:

FIG. 1 (A) shows part of the human APP target RNA sequence (SEQ ID NO:1) from 5′ to 3′ with the target adenosine underlined and in bold. (B) shows the sequence of the six oligonucleotides that were initially tested (EON 1 to 6; from 5′ to 3′), of which EON 1 to 4 contain a 2′,2′-difluoro (diF) substitution at a variety of nucleotide positions as indicated. EON 7 to 10 are shorter versions of these initial EONs and were tested in subsequent experiments. EON 1 to 10 have SEQ ID NO:2 to 11, respectively. The positions are incrementing positively (+) towards the 5′ terminus of the oligonucleotide, and negatively (−) incremented towards the 3′ terminus of the oligonucleotide, wherein the orphan nucleotide (the orphan cytidine) is at position 0. C_{f2}=cytidine carrying a diF substitution at the 2′ position of the ribose. T, C and A nucleosides (upper case, bold) are DNA. Underlined A, U, and G nucleosides (not bold) are 2′-MOE modified. Underlined C nucleosides (not bold) are 2′-MOE modified 5-methyl-C. c, a, u, and g nucleosides (lower case, not bold, not underlined) are 2′-OMe modified. The asterisk depicts a phosphorothioate (PS) linkage. The “\hat{ }” symbol refers to a methylphosphonate (MP) linkage. The triplet with the orphan nucleotide in the middle (position 0) and its two surrounding nucleotides is given by a grey box. (C) shows the results of a biochemical editing assay in which EONs 1 to 6 were tested for deamination of a target adenosine in a hAPP target RNA sequence.

FIG. 2 shows the percentage of editing in human ARPE-19 cells using oligonucleotides EON 1 to 5 of FIG. 1B after gymnotic uptake (=no transfection), in comparison to a non-treated control (NT).

FIG. 3 shows the percentage of editing in human ARPE-19 cells using EON 7 to 10 of FIG. 1B after gymnotic uptake, in comparison to a non-treated control (NT).

FIG. 4 shows the percentage of editing in human primary hepatocytes using EON 7 to 10 of FIG. 1B after gymnotic uptake, in comparison to a non-treated control (NT).

FIG. 5 (A) shows part of the mouse APP target RNA sequence (SEQ ID NO: 12) from 5′ to 3′ with the target adenosine underlined and in bold. (B) shows the sequence of two oligonucleotides that were initially tested (EON A and B; from 5′ to 3′; SEQ ID NO: 13 and 14, respectively), of which EON B contains a diF substitution at position 0 (=the orphan nucleotide). C_{f2}=cytidine carrying a diF substitution at the 2′ position of the ribose. C and A nucleosides (upper case, bold) are DNA. Underlined A nucleosides (not bold) are 2′-MOE modified. Underlined C nucleosides (not bold) are 2′-MOE modified 5-methyl-C. c, a, u, and g nucleosides (lower case, not bold, not underlined) are 2′-OMe modified. Gf, Uf, Af, and Cf are nucleotides carrying a single 2′-fluoro substitution in the ribose moiety. The asterisk depicts a phosphorothioate (PS) linkage. The “\hat{ }” symbol refers to a methylphosphonate (MP) linkage. The symbol “!” refers to a (1,3-dimethylimidazolidin-2-ylidene) phosphoramidate linkage, generally referred to as “PNdmi”. The triplet with the orphan nucleotide in the middle (position 0) and its two surrounding nucleotides is given by a grey box. (C) shows the percentage of editing in mouse RPE cells after gymnotic uptake of EON A and B respectively, in comparison to a negative control.

FIG. 6 shows in (A) the sequence of the Homo sapiens (pre-) mRNAs surrounding the codon (CAG in bold) encoding the glutamine at position 152 in the PCSK9 proprotein from 5′ to 3′ (SEQ ID NO:15). In (B) the sequences of the four guide oligonucleotides are given (5′ to 3′; SEQ ID NO:16 to 19, respectively). Oligonucleotides with the name PCSK9-18 and -24 are complementary to the human PCSK9 sequence shown in (A). PCSK9-32, and -34 are complementary to the monkey PCSK9 sequence. The chemical modifications in each of the AONs are provided as follows: an asterisk indicates a PS linkage between two nucleosides; lower case nucleotides are modified with 2′-OMe; upper-case italic nucleotides are modified with 2′-MOE, wherein C represents a 2′-MOE modified 5-methyl-C; upper-case underlined nucleotides are modified with 2′-F; I is deoxyinosine (opposite a C in the target sequence). Z means a DNA nucleotide carrying a Benner's base (a cytidine analog as disclosed in WO 2020/252376). C_{f2} is cytidine carrying a 2′,2′-difluoro substitution in the ribose moiety. The linkage 3′ from I is a methylphosphonate (MP) linkage given by the “\hat{ }” symbol; the “!” symbol indicates a phosphoramidate (PNdmi) linkage.

FIG. 7 shows the percentage A to I editing of endogenous (human) PCSK9 target RNA in human Hela cells, after transfection with the four oligonucleotides of FIG. 6 as indicated. Negative controls were i) a non-treated (NT) sample, ii) transfection with a scrambled unrelated oligonucleotide (not shown), and iii) a mock transfection (not shown).

FIG. 8 shows the percentage A to I editing of endogenous (human) PCSK9 target RNA in human hepatocytes after gymnotic incubation with 10 UM of the four oligonucleotides of FIG. 6 as indicated.

### DETAILED DESCRIPTION

There is a constant need for improving the pharmacokinetic properties of RNA-editing antisense oligonucleotides (AONs, sometimes referred to as ‘editing oligonucleotides’, or ‘EONs’) without negatively affecting editing efficiency of the target adenosine in the target RNA. Many chemical modifications exist and may be applied in the generation of AONs, whose properties are not always compatible with the desire of achieving efficient RNA editing.

ADARs are multidomain proteins with N-terminal double stranded RNA binding domains (dsRBDs) and C-terminal deaminase domains. Two ADAR genes encode catalytically active ADARs in humans (ADAR encoding ADAR1 proteins and ADARB1 encoding the ADAR2 protein). ADAR1 is expressed in two protein isoforms (p110 and p150) that differ in their N-terminal structures. Since the substrate for ADARs is an RNA duplex, the enzymes access the reactive adenosine using a base flipping mechanism (Stephens O. M. et al. Biochemistry. 2000. 39 (40): 12243-12251). Also, because ADARs require duplex RNA for activity, their reaction can be directed to specific adenosines in different transcripts using complementary guide strands for duplex formation at the target sites. This approach is currently being pursued to develop therapeutic guide strands that recruit ADARs to correct disease-causing mutations in RNA (Qu L. et al. Nat. Biotechnol. 2019. 37 (9): 1059-1069; Merkle T. et al. Nat. Biotechnol. 2019. 37 (2): 133-138; Katrekar D. et al. Nat. Methods 2019. 16 (3): 239-242; Monian P. et al. Nat. Biotechnol. 2022: p. doi: 10.1038/s41587-022-01225-1). While this approach is promising, ADARs have sequence preferences that make certain adenosines disfavored for reaction, limiting the current scope of this approach. For instance, the nearest neighbor nucleotide preferences for ADARs show a strong bias against reaction at adenosines in 5′-GA sites (Eggington J. M. et al. Nat. Commun. 2011. 2 (319)). This preference is explained by structural studies of ADAR2 bound to transition state analog-containing RNA that suggest a clash between the 2-amino group of the 5′-G and G489 of the ADAR2 loop involved in stabilizing the flipped-out conformation required for the adenosine deamination reaction (Matthews et al. 2016). Earlier work with fusion proteins bearing ADAR deaminase domains indicated that editing efficiency at 5′-GA sites could be improved with a G-A or G-G pair at the 5′ nearest neighbor (Schneider M. F. et al. Nucleic Acids Res. 2014. 42 (10): p.e87).

Mutagenesis studies of human ADAR2 revealed that a single mutation at residue 488 from glutamate to glutamine (E488Q), gave an increase in the rate constant of deamination by 60-fold when compared to the wild-type enzyme (Kuttan and Bass. Proc Natl Acad Sci USA 2012. 109 (48): 3295-3304). During the deamination reaction, ADAR flips the edited base out of its RNA duplex, and into the enzyme active site (Matthews et al. 2016). When ADAR2 edits adenosines in the preferred context (an A: C mismatch) the nucleotide opposite the target adenosine is often also referred to as the ‘orphan cytidine’. The crystal structure of ADAR2 E488Q bound to double stranded RNA (dsRNA) revealed that the glutamine (Gln) side chain at position 488 can donate an H-bond to the N3 position of the orphan cytidine, which leads to the increased catalytic rate of ADAR2 E488Q. In the wild-type enzyme, wherein a glutamate (Glu) is present at position 488 instead of a glutamine (Gln) the amide group of the glutamine is absent and is instead a carboxylic acid. To obtain the same contact of the orphan cytidine with the E488Q mutant would then, for the wild-type situation, require protonation for this contact to occur. To make use of endogenously expressed ADAR2 to correct disease relevant mutations, it is essential to maximize the editing efficiency of the wild type ADAR2 enzyme present in the cell. WO2020/252376 discloses the use of AONs with modified RNA bases, especially at the position of the orphan cytidine to mimic the hydrogen-bonding pattern observed by the E488Q ADAR2 mutant. By replacing the nucleotide opposite the target adenosine in the AON with cytidine analogs that serve as H-bond donors at N3, it was envisioned that it would be possible to stabilize the same contact that is believed to provide the increase in catalytic rate for the mutant enzyme. Two cytidine analogs were of particular interest: pseudoisocytidine (also referred to as ‘piC’; Lu et al. J Org Chem 2009. 74 (21): 8021-8030; Burchenal et al. (1976) Cancer Res 36:1520-1523) and Benner's base Z (also referred to as ‘dZ’ or 6-amino-5-nitro-2(1H)-pyridone; Yang et al. Nucl Acid Res 2006. 34 (21): 6095-6101) that were initially selected because they offer hydrogen-bond donation at N3 with minimal perturbation to the shape of the nucleobase.

It was initially thought that the presence of the cytidine analog in the AON may exist in addition to modifications to the ribose 2′ group. The ribose 2′ groups in the AON can be independently selected from 2′-H (i.e., DNA), 2′-OH (i.e., RNA), 2′-O-Me, 2′-MOE, 2′-F, or 2′-4′-linked (i.e., a bridged nucleic acid such as a locked nucleic acid (LNA)), or other 2′ substitutions. The 2′-4′ linkage can be selected from linkers known in the art, such as a methylene linker or constrained ethyl linker. However, the inventors of the present invention realized that a certain alternative chemical modification of the sugar moiety at the 2′ position could potentially increase the EON RNA editing inducing effect even further and even in the absence of the Benner's base Z (hence, with a normal cytosine nitrogenous base) at the orphan nucleotide position. Generally spoken, the invention as disclosed herein relates to an AON that can form a double stranded nucleic acid complex with a target RNA molecule, wherein the double stranded nucleic acid complex can recruit an (endogenous) adenosine deaminating enzyme, such as (human) ADAR2, for deamination of a target adenosine in the target RNA molecule, wherein the orphan nucleotide in the AON comprises a 2′,2′-difluoro (diF) or a 2′-fluoro-2-C-2′-methyl modification in the sugar moiety. In one embodiment, the AON is a (single-stranded) AON that targets a pre-mRNA or an mRNA, wherein the target nucleotide in the target (pre-) mRNA molecule is an adenosine, wherein the AON when hybridized to the target RNA molecule can (as a double stranded complex) recruit an (endogenous) adenosine deaminating enzyme, and wherein then the adenosine is deaminated to an inosine, which is being read as a guanosine by the translation machinery. In another embodiment, the AON is hybridized to a complementary (sense) strand, that may comprise a different set of chemical modifications and that together with the AON forms a heteroduplex complex. In another embodiment, the AON, or the complementary strand to which it may be attached, is linked to a delivery-enhancing moiety, such as a GalNAc complex when the target cell is a liver cell for instance. Importantly, the editing events do not have to be for the purpose of repairing a mutation (such as the de novo occurrence of a TGA or TAG stop codon in the DNA, and the respective editing to a UGI or UIG codon in the target RNA transcript, being read by the translation machinery as UGG) but may also be applied for gain of function or loss of function purposes. For example, changing Asp is useful for changing signal sequence sites (to prevent entry into the secretory pathway and/or plasma membrane trafficking and/or secretion) or caspase cleavage sites (to prevent cleavage by caspases) to give an example. Changing Ser and Thr may be useful to prevent phosphorylation of these residues; in some cases, where these residues are so-called master regulators of phosphorylation, this may have major (desirable) effects on protein phosphorylation patterns of the protein in question. Changing a Lys is useful to prevent sumoylation, thereby preventing degradation of a protein. Making amino acid changes, including the ones described here, may be useful to modulate protein-protein interactions with major impact on protein localization, activation, or function of the protein in question or its interaction partner, potentially affecting entire pathways. Many of these kinds of changes and their effect on protein interactions or post-translational modifications are known from the literature and specialized databases and the person skilled in the art would readily know how to make these changes through RNA editing.

The invention also relates to an AON according to the invention that can form a double stranded nucleic acid complex with a target RNA molecule, wherein the double stranded nucleic acid complex can recruit an adenosine deaminating enzyme for deamination of a target adenosine in the target RNA molecule, wherein the nucleotide directly 5′ of the target adenosine in the target RNA molecule is a guanosine, and wherein the nucleotide in the AON that is opposite the guanosine is a nucleotide analog that can induce a syn conformation of the guanosine. A preferred nucleotide analog at that position is 3-deaza dA (see U.S. 63/389,441; unpublished).

In one embodiment, at least one nucleotide in the AON besides the orphan nucleotide comprises an arabinose. In one embodiment, at least one nucleotide in the AON is a Xeno Nucleic Acid (XNA), such as a bridged nucleic acid (e.g. Locked Nucleic Acid (LNA), constrained ethyl (cEt), and amido-bridged nucleic acid (AmNA), a tricyclo DNA (tcDNA), an alpha-anomeric bicyclic DNA (abcDNA), a 2′-Fluoro-Arabino Nucleic Acid (FANA), a Glycol Nucleic Acid (GNA), a Threose Nucleic Acid (TNA), a 1,5-anhydrohexitol Nucleic Acid (HNA), a Cyclohexene Nucleic Acid (CeNA), a phosphorodiamidate morpholino (PMO), a L-acyclic threoninol nucleic acid (L-aTNA), a D-acyclic threoninol nucleic acid (D-aTNA), an unlocked nucleic acid (UNA), a serinol nucleic acid (SNA) and a Peptide Nucleic Acid (PNA); or derivatives thereof. In one embodiment, the AON comprises one or more combinations of these XNA's.

The AON according to the invention can comprise internucleoside linkage modifications. In one embodiment one such other internucleoside linkage can be a phosphonoacetate, phosphorothioate (PS), a methylphosphonate (MP), a PNdmi linkage, a phosphorodithioate, a (substituted) guanidine phosphoramidate linkage, or a sulfonylphosphoramidate (such as e.g., mesyl phosphoramidate, or 4-acetamidobenzenesulfonyl phosphoramidate). A preferred linkage is a PS linkage. Preferred positions for MP linkages are described in WO2020/201144. In one embodiment, the internucleotide linkage can be a phosphodiester wherein the OH group of the phosphodiester has been replaced by alkyl, alkoxy, aryl, alkylthio, acyl, —NR1R1, alkenyloxy, alkynyloxy, alkenylthio, alkynylthio, —S—Z+, —Se—Z+, or —BH3-Z+, and wherein R1 is independently hydrogen, alkyl, alkenyl, alkynyl, or aryl, and wherein Z+ is ammonium ion, alkylammonium ion, heteroaromatic iminium ion, or heterocyclic iminium ion, any of which is primary, secondary, tertiary or quaternary, or Z is a mono- or divalent metal ion, and is preferably a PS linkage. In one embodiment, the AON comprises stereo defined (also referred to as chirally defined) linkages, including the ones described in WO2011/005761, WO2014/010250, WO2014/012081, WO2015/107425, WO2017/015575, WO2017/062862, WO2017/160741, WO2017/192664, WO2017/192679, WO2017/198775, WO2017/210647, WO2018/067973, WO2018/098264, WO2018/223056, WO2018/223073, WO2018/223081, WO2018/237194, WO2019/032607, WO2019/055951, WO2019/075357, WO2019/200185, WO2019/217784, WO2019/219581, WO2020/118246, WO2020/160336, WO2020/191252, WO2020/196662, WO2020/219981, WO2020/219983, WO2020/227691, WO2021/071788, WO2021/071858, WO2021/178237, WO2021/234459, WO2021/237223, and WO2022/099159.

The orphan nucleotide (the nucleotide directly opposite the target adenosine), as outlined in the art, generally comprises a ribose with a 2′-OH group, a deoxyribose with a 2′-H group, a deoxyribose with a 2′-F group (2′-F), an arabinose, a 2′-deoxy-2′-fluoroarabinose (FANA), and preferably does not comprise a ribose carrying a 2′-O-Me modification or a 2′-MOE modification. However, according to the present invention the orphan nucleotide comprises a deoxyribose that is a 2′,2′-disubstituted ribose, with substituents being selected from OH, F and CH₃. In the case of 2′,2′-difluoro, this is referred to as difluoro (diF). Further, the AON of the present invention may comprise 2′-OMe, 2′-MOE and/or 2′-F modifications at other positions within the AON.

In one embodiment, the invention relates to a method for the deamination of at least one target adenosine present in a target RNA molecule in a cell, the method comprising the steps of providing the cell with an AON according to a first aspect of the invention, or a composition according to a second aspect of the invention, allowing uptake by the cell of the AON, allowing annealing of the AON to the target RNA molecule, allowing a mammalian enzyme with nucleotide deaminase activity to deaminate the target nucleotide in the target RNA molecule, and optionally identifying the presence of the deaminated nucleotide in the target RNA molecule. Preferably, the presence of the target RNA molecule is detected by either (i) sequencing the target sequence, (ii) assessing the presence of a functional, elongated, full length and/or wild type protein when the target adenosine is in a stop codon, or (iii) using a functional read-out, wherein the target RNA after the deamination encodes a functional, full length, elongated and/or wild type protein. The present invention therefore also relates to AONs that target premature termination stop codons (PTCs) present in the (pre-) mRNA to alter the adenosine present in the stop codon to an inosine (read as a G), which in turn then results in read-through during translation and a full-length functional protein. The teaching of the present invention, as outlined herein, is applicable for all genetic diseases that may be targeted with AONs and may be treated through RNA editing. However, it is also applicable to ‘loss of function’ editing, or in another embodiment a ‘gain-of-function’ editing in which the deamination of the adenosine causes a gain of function, for instance the introduction of another secondary protein structure or the introduction of a functional site, for instance a phosphorylation site.

In one embodiment, the AON according to the invention comprises 2, 3, 4, 5, 6, 7, 8, 9 or 10 mismatches, wobbles and/or bulges with the complementary target RNA region. When the nucleotide opposite the target adenosine is a cytidine, which then carries a diF substitution at the 2′ position of the ribose sugar moiety, as further outlined herein, or a cytidine analog as for example disclosed in WO2020/252376, the AON mismatches at least once with the target RNA molecule. However, in certain embodiments it is preferred to position a U opposite the target adenosine, which is in principle not a mismatch. In a preferred aspect one or more additional mismatching nucleotides, wobbles and/or bulges are present between AON and target RNA. These should add to the RNA editing efficiency by the ADAR present in the cell, at the target adenosine position. The person skilled in the art can determine whether hybridization under physiological conditions still does take place. The AON of the present invention can recruit (engage) a mammalian adenosine deaminating enzyme present in the cell. This is preferably an ADAR enzyme, more preferably ADAR1 or ADAR2. However, when the target RNA molecule is a tRNA, the adenosine deaminating enzyme may also be an Adenosine Deaminase acting on tRNA (ADAT). The AONs according to the present invention can utilise endogenous cellular pathways and naturally available ADAR enzymes, or enzymes with ADAR activity (which may be yet unidentified ADAR-like enzymes) to specifically edit a target adenosine in a target RNA sequence. As disclosed herein, the single-stranded AONs of the invention can bring about deamination of a specific target, such as adenosine, in a target RNA molecule, wherein the 5′ neighbouring nucleotide from the adenosine is a guanosine. Ideally, at least one target nucleotide is deaminated. Alternatively, 1, 2, or 3 further nucleotides are deaminated. This means that a single AON according to the invention may be applied for deamination of a target adenosine, but also for deamination further up- or downstream of the target adenosine. The orphan nucleotide according to the present invention may be within an AON that brings about RNA editing, wherein the AON comprises a stem-loop structure (a self-looping hairpin structure) as disclosed for instance in WO2016/097212, WO2017/010556, WO2017/050306, WO2019/111957, WO2020/001793, WO2021/113270, WO2021/243023, WO2022/078995, or wherein the AON is fully or almost fully complementary to the target RNA molecule's sequence, and does not necessarily comprise a hairpin structure, such as for instance disclosed in WO2017/220751 and WO2018/041973. Notably, the AON of the present invention may also be linked to recombinant deaminase domains, as was shown by Montiel-Gonzalez et al. (2013) and Vogel et al. (2014), because such ‘early’ technologies in which oligonucleotide sequences were used to bring about RNA editing may also benefit from the introduction of an orphan nucleotide as disclosed herein and as further outlined in the present invention. Hence, the AONs of the present invention are not limited in the sense that the AON does not have a stem-loop structure, or that the AON is not linked to a deaminase or deaminase domain. Nevertheless, it is preferred that the AON is relatively short such that cell entry is feasible and the AON is therapeutically relevant when administered in a ‘naked’ form, and therefore preferably does not have a stem-loop structure and preferably is not linked to deaminase moieties. However, the AON of the present invention may be linked to other moieties that may enhance cellular uptake or cellular trafficking towards the site of action. Examples are oligonucleotides that are conjugated to GalNAc ligands, such as those described in WO2014/179620 and WO2017/079745, which improve the delivery of oligonucleotides in vivo, especially to liver cells. Another example is formed by the group of saponins that can be conjugated with the oligonucleotide, and which improve cellular entry and trafficking (WO2020/126626; WO2021/122998; Wang M. et al. Drug Design, Development and Therapy. 2018. 12:3705-3715). In one embodiment, the AON of the present invention, or the complementary sense oligonucleotide to which the AON may be hybridized before cell entry, is conjugated to one or more GalNAc ligands and/or to a saponin. The skilled person is aware of what ligands and what saponins may be best used for what therapeutic use.

The invention relates to an antisense oligonucleotide (AON) that can form a double stranded nucleic acid complex with a target RNA molecule, wherein the double stranded nucleic acid complex can recruit an adenosine deaminating enzyme for deamination of a target adenosine in the target RNA molecule, wherein the nucleotide in the AON that is opposite the target adenosine is the orphan nucleotide, and wherein the orphan nucleotide has the structure of formula I:

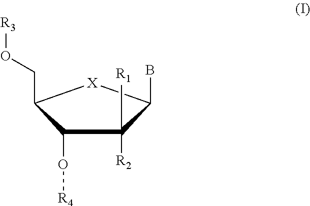

wherein: X is O, NH, CH₂, Se, or S; B is a nitrogenous base selected from the group consisting of: cytosine, uracil, isouracil, N3-glycosylated uracil, pseudoisocytosine, 8-oxo-adenine, and 6-amino-5-nitro-2(1H)-pyridone; R₁ and R₂ are both selected, independently, from OH, fluoro (F) or CH₃; R₃ is the part of the AON that is 5′ of the orphan nucleotide, consisting of 7 to 30 nucleotides; and R₄ is the part of the AON that is 3′ of the orphan nucleotide, consisting of 4 to 25 nucleotides. In one embodiment R₁ or R₂ is F and R₁ or R₂ is either CH₃ or also F. In another embodiment R₁ and R₂ are both F. In another embodiment, one or more nucleotides in R₃ and/or R₄ comprise a chemical modification that is a mono- or di-substitution at the 2′,3′ and/or 5′ position of the ribose sugar, selected from the group consisting of: —OH; —F; substituted or unsubstituted, linear or branched lower (C₁-C₁₀) alkyl, alkenyl, alkynyl, alkaryl, allyl, or aralkyl, that may be interrupted by one or more heteroatoms; —O—, S—, or N-alkyl; —O—, S—, or N-alkenyl; —O—, S—, or N-alkynyl; —O—, S—, or N-allyl; —O-alkyl-O-alkyl; -methoxy; -aminopropoxy; -methoxyethoxy; -dimethylamino oxyethoxy; and -dimethylaminoethoxyethoxy.

In one embodiment, the AON according to the present invention comprises at least one phosphonoacetate, phosphorothioate (PS), a methylphosphonate (MP), a phosphorodithioate (PS2), a guanidine phosphoramidate linkage, a PNdmi linkage, or a sulfonylphosphoramidate (such as e.g., mesyl phosphoramidate, or 4-acetamidobenzenesulfonyl phosphoramidate). In a preferred embodiment, the double stranded nucleic acid complex can recruit an endogenous ADAR enzyme, preferably wherein the ADAR enzyme is an endogenous ADAR2 enzyme. The double stranded AON/target RNA molecule complex interacts through Watson-Crick base-pairing, except at the position of the 5′-G and generally also not at the position of the target adenosine which is opposite the orphan nucleotide. The length of the AON may vary depending on the structures that are present (hairpin structured AONs are generally longer, but when no hairpin structure is present, the AON may be relatively ‘short’, preferably comprising approximately 15 to 25 nucleotides). The AON of the present invention does not necessarily carry a recruiting portion (such as a hairpin or a stem-loop structure) to attract ADAR, but it is not excluded. In one embodiment, the AON according to the invention consists of 15, 16, 17, 18, 19, 20, 21, 22, 23, 24, 25, 26, 27, 28, 29, 30, 31, 32, 33, 34, 35, 36, 37 or 38 nucleotides.

The invention also relates to a pharmaceutical composition comprising an AON according to the invention, and a pharmaceutically acceptable carrier or diluent.

In one embodiment, the invention relates to an AON according to the invention, or a pharmaceutical composition according to the invention, for use in the treatment, amelioration, slowing down progression, or prevention of a genetic disease caused by a premature termination codon.

The invention also relates to a method for the deamination of at least one target adenosine present in a target RNA molecule in a cell, the method comprising the steps of: (i) providing the cell with an AON according to the invention, or a pharmaceutical composition according to the invention; (ii) allowing annealing of the AON to the target RNA molecule to form a double stranded nucleic acid complex capable of recruiting an adenosine deaminating enzyme in the cell, preferably an endogenous adenosine deaminating enzyme, more preferably ADAR2; (iii) allowing the adenosine deaminating enzyme to deaminate the target adenosine in the target RNA molecule; and (iv) optionally identifying the presence of the deaminated adenosine in the target RNA molecule. In one embodiment, step (iv) comprises: (a) sequencing a region of the target RNA molecule, wherein the region comprises the deaminated target adenosine; (b) assessing the presence of a functional, elongated, full length and/or wild type protein when the target adenosine is in a stop codon; or (c) using a functional read-out, wherein the target RNA molecule after the deamination encodes a functional, full length, elongated and/or wild type protein.

In one embodiment, an in vitro, ex vivo, or in vivo method is provided for the deamination of at least one target adenosine present in a target RNA molecule, the method comprising the steps of: (i) providing an AON that can form a double stranded nucleic acid complex with a target RNA molecule, wherein the double stranded nucleic acid complex is capable of recruiting an adenosine deaminating enzyme for deamination of a target adenosine in the target RNA molecule, wherein the nucleotide in the AON that is opposite the target adenosine is the orphan nucleotide, and wherein the orphan nucleotide has the structure of formula I:

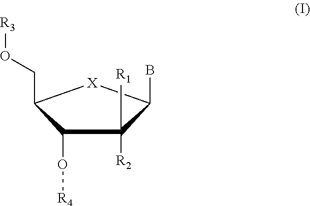

wherein: X is O, NH, CH₂, Se, or S; B is a nitrogenous base selected from the group consisting of: cytosine, uracil, isouracil, N3-glycosylated uracil, pseudoisocytosine, 8-oxo-adenine, and 6-amino-5-nitro-2(1H)-pyridone; R₁ and R₂ are both selected, independently, from OH, fluoro (F) or CH₃; R₃ is the part of the AON that is 5′ of the orphan nucleotide, consisting of 7 to 30 nucleotides; and R₄ is the part of the AON that is 3′ of the orphan nucleotide, consisting of 4 to 25 nucleotides; (ii) allowing annealing of the AON to the target RNA molecule to form a double stranded nucleic acid complex with the target RNA molecule; (iii) allowing a mammalian adenosine deaminating enzyme to deaminate the target adenosine in the target RNA molecule; and (iv) optionally identifying the presence of the deaminated adenosine in the target RNA molecule. Preferably, the adenosine deaminating enzyme is an endogenous ADAR enzyme, even more preferably ADAR2.

In one embodiment, the invention relates to an AON according to the invention, or a pharmaceutical composition according to the invention, for use in the treatment or prevention of a genetic disorder, preferably selected from the group consisting of: Hurler Syndrome, alpha-1-antitrypsin (A1AT) deficiency, (familial) hypercholesterolemia, Parkinson's disease, Rett syndrome, Stargardt Disease, Citrullinemia Type 1, autosomal recessive non-syndromic hearing loss, X-linked retinoschisis, argininosuccinate lyase deficiency, Duchenne/Becker muscular dystrophy, Non-Alcoholic Steatohepatitis (NASH), Myotonic dystrophy type I, Myotonic dystrophy type II, Huntington's disease, Usher syndrome (such as Usher syndrome type I, II, and III), Charcot-Marie-Tooth disease, Cystic fibrosis, Alzheimer's disease, albinism, Amyotrophic lateral sclerosis, Asthma, β-thalassemia, Epileptic Encephalopathy, CADASIL syndrome, Chronic Obstructive Pulmonary Disease (COPD), Distal Spinal Muscular Atrophy (DSMA), Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hereditary Cancer predisposing Syndrome, Hunter Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anaemia, Spinal Muscular Atrophy, Tay-Sachs Disease, X-linked immunodeficiency, Sturge-Weber Syndrome, and cancer, such as breast and lung cancer.

In yet another embodiment, the invention relates to an AON capable of forming a double stranded nucleic acid complex with a target RNA molecule, for use in the treatment of a genetic disorder, wherein the double stranded nucleic acid complex can recruit an adenosine deaminating enzyme for deamination of a target adenosine in the target RNA molecule.

In yet another embodiment, the invention relates to an AON capable of forming a double stranded nucleic acid complex with a target RNA molecule, for use in the treatment of a disorder, wherein the double stranded nucleic acid complex is capable of recruiting an adenosine deaminating enzyme for deamination of a target adenosine in the target RNA molecule, and wherein the disorder is not caused by a mutation, but wherein the use is for a gain-of-function purpose to alleviate, treat, prevent, or ameliorate the disease. In another embodiment, the invention relates to a method of treating a subject, preferably a human subject in need thereof, wherein the subject suffers from a genetic disorder caused by a mutation resulting in a premature termination codon.

In one embodiment, a method comprises the steps of administering to the subject an AON or pharmaceutical composition according to the invention, allowing the formation of a double stranded nucleic acid complex of the AON with its specific complementary target nucleic acid in a cell in the subject; allowing the engagement of an endogenous present adenosine deaminating enzyme, such as ADAR2; and allowing the enzyme to deaminate the target adenosine in the target nucleic target molecule to an inosine, thereby alleviating, preventing or ameliorating the genetic disease. The genetic diseases that may be treated according to this method are preferably, but not limited to the genetic diseases listed herein, and any other disease in which deamination of a specific adenosine would be beneficial for a patient in need thereof.

The skilled person knows that an oligonucleotide, such as an RNA oligonucleotide, generally consists of repeating monomers. Such a monomer is most often a nucleotide or a chemically modified nucleotide. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide. A modification in the pentose sugar is therefore often referred to as a “scaffold modification”. The original pentose sugar may be replaced in its entirety by another moiety that similarly connects the base and the phosphate. It is therefore understood that while a pentose sugar is often a scaffold, a scaffold is not necessarily a pentose sugar. Examples of scaffold modifications that may be applied in the monomers of the AONs of the present invention are disclosed in WO2020/154342, WO2020/154343, and WO2020/154344. A base, sometimes called a nucleobase, is generally adenine, cytosine, guanine, thymine or uracil, or a derivative thereof. A base, sometimes called a nucleobase, is defined as a moiety that can bond to another nucleobase through H-bonds, polarized bonds (such as through CF moieties) or aromatic electronic interactions. Cytosine, thymine, and uracil are pyrimidine bases, and are generally linked to the scaffold through their 1-nitrogen. Adenine and guanine are purine bases and are generally linked to the scaffold through their 9-nitrogen.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked scaffolds of an oligonucleotide is often called the “backbone” of the oligonucleotide. Because phosphodiester bonds connect neighboring monomers together, they are often referred to as “backbone linkages”. It is understood that when a phosphate group is modified so that it is instead an analogous moiety such as a phosphorothioate (PS), such a moiety is still referred to as the backbone linkage of the monomer. This is referred to as a “backbone linkage modification”. In general terms, the backbone of an oligonucleotide comprises alternating scaffolds and backbone linkages.

The nucleobases in an AON of the present invention can be adenine, cytosine, guanine, thymine, or uracil or any other moiety able to interact with another nucleobase through H-bonds, polarized bonds (such as CF) or aromatic electronic interactions. The nucleobases at any position in the AON of the present invention can be a modified form of adenine, cytosine, guanine, or uracil, such as hypoxanthine (the nucleobase in inosine), pseudouracil, pseudocytosine, isouracil, N3-glycosylated uracil, 1-methylpseudouracil, orotic acid, agmatidine, lysidine, 2-thiouracil, 2-thiothymine, 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine, 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, 8-oxo-adenine, pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose). The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy) ribosyl sugar.

In an embodiment, the nucleotide analog is an analog of a nucleic acid nucleotide. In an embodiment, the nucleotide analog is an analog of adenosine, guanosine, cytidine, thymidine, uridine, deoxyadenosine, deoxyguanosine, deoxycytidine, deoxythymidine or deoxyuridine. In an embodiment, the nucleotide analog is not guanosine or deoxyguanosine. In an embodiment, the nucleotide analog is not a nucleic acid nucleotide. In an embodiment, the nucleotide is not adenosine, guanosine, cytidine, thymidine, uridine, deoxyadenosine, deoxyguanosine, deoxycytidine, deoxythymidine or deoxyuridine.

In one embodiment, the AON of the present invention may comprise one or more nucleotides carrying a 2′-O-(2-methoxy)ethyl (2′-MOE) ribose modification. Also, in one embodiment, the AON comprises one or more nucleotides not carrying a 2′-MOE ribose modification, and wherein the 2′-MOE ribose modifications are at positions that do not prevent the enzyme with adenosine deaminase activity from deaminating the target adenosine. In another embodiment, the AON comprises 2′-O-methyl (2′-O-Me) ribose modifications at the positions that do not comprise a 2′-MOE ribose modification, and/or wherein the oligonucleotide comprises deoxynucleotides at positions that do not comprise a 2′-MOE ribose modification. In one embodiment the AON comprises one or more nucleotides comprising a 2′ position comprising a 2′-MOE, 2′-O-Me, 2′-OH, 2′-deoxy, 2′-fluoro (2′-F), 2′,2′-difluoro (diF) modification, 2′-fluoro-2′-C-methyl modification, or a 2′-4′-linkage (i.e., a bridged nucleic acid such as a locked nucleic acid (LNA)). In another embodiment, other nucleic acid monomer that are applied are arabinonucleic acids and 2′-deoxy-2′-fluoroarabinonucleic acid (FANA), for instance for improved affinity purposes. The 2′-4′ linkage can be selected from linkers known in the art, such as a methylene linker or constrained ethyl linker. A wide variety of 2′ modifications are known in the art. Further examples are disclosed in further detail in WO2016/097212, WO2017/220751, WO2018/041973, WO2018/134301, WO2019/219581, WO2019/158475, and WO2022/099159 for instance. In all cases, the modifications should be compatible with editing such that the oligonucleotide fulfils its role as an editing oligonucleotide. Where a monomer comprises an unlocked nucleic acid (UNA) ribose modification, that monomer can have a 2′ position comprising the same modifications discussed above, such as a 2′-MOE, a 2′-O-Me, a 2′-OH, a 2′-deoxy, a 2′-F, a 2′,2′-diF, a 2′-fluoro-2′-C-methyl, an arabinonucleic acid, a FANA, or a 2′-4′-linkage (i.e., a bridged nucleic acids such as a locked nucleic acid (LNA)). Again, in all cases, the modifications should be compatible with editing such that the oligonucleotide fulfils its role as an AON that can, when attached to its target sequence recruit an adenosine deaminase enzyme. In all aspects of the invention, the enzyme with adenosine deaminase activity is preferably ADAR1, ADAR2, or ADAT. In a highly preferred embodiment, the AON is an RNA editing oligonucleotide that targets a pre-mRNA or an mRNA, wherein the target nucleotide is an adenosine in the target RNA, wherein the adenosine is deaminated to an inosine, which is being read as a guanosine by the translation machinery. The invention also relates to a pharmaceutical composition comprising the AON as characterized herein, and a pharmaceutically acceptable carrier.

The term ‘nucleoside’ refers to the nucleobase linked to the (deoxy) ribosyl sugar, without phosphate groups. A ‘nucleotide’ is composed of a nucleoside and one or more phosphate groups. The term ‘nucleotide’ thus refers to the respective nucleobase-(deoxy) ribosyl-phospholinker, as well as any chemical modifications of the ribose moiety or the phospho group. Thus, the term would include a nucleotide including a locked ribosyl moiety (comprising a 2′-4′ bridge, comprising a methylene group or any other group), an unlocked nucleic acid (UNA), a nucleotide including a linker comprising a phosphodiester, phosphonoacetate, phosphotriester, PS, phosphoro(di)thioate, MP, phosphoramidate linkages, and the like. Sometimes the terms adenosine and adenine, guanosine and guanine, cytidine and cytosine, uracil and uridine, thymine and thymidine/uridine, inosine and hypoxanthine, are used interchangeably to refer to the corresponding nucleobase on the one hand, and the nucleoside or nucleotide on the other. Sometimes the terms nucleobase, nucleoside and nucleotide are used interchangeably, unless the context clearly requires differently, for instance when a nucleoside is linked to a neighbouring nucleoside and the linkage between these nucleosides is modified. As stated above, a nucleotide is a nucleoside+one or more phosphate groups. The terms ‘ribonucleoside’ and ‘deoxyribonucleoside’, or ‘ribose’ and ‘deoxyribose’ are as used in the art. Whenever reference is made to an oligonucleotide, oligo, ON, ASO, oligonucleotide composition, antisense oligonucleotide, AON, (RNA) editing oligonucleotide, EON, and RNA (antisense) oligonucleotide both oligoribonucleotides and deoxyoligoribonucleotides are meant unless the context dictates otherwise. Whenever reference is made to an ‘oligoribonucleotide’ it may comprise the bases A, G, C, U or I. Whenever reference is made to a ‘deoxyoligoribonucleotide’ it may comprise the bases A, G, C, T or I. However, an AON of the present invention may comprise a mix of ribonucleosides and deoxyribonucleosides. When a deoxyribonucleoside is used, hence without a modification at the 2′ position of the sugar, the nucleotide is often abbreviated to dA. dC, dG or T in which the ‘d’ represents the deoxy nature of the nucleoside, while a ribonucleoside that is either normal RNA or modified at the 2′ position is often abbreviated without the ‘d’, and often abbreviated with their respective modifications and as explained herein.

Whenever reference is made to nucleotides in the oligonucleotide construct, such as cytosine, 5-methylcytosine, 5-hydroxymethylcytosine, 5-formylcytosine, 5-acetylcytosine, 5-hydroxycytosine, and β-D-glucosyl-5-hydroxymethylcytosine are included; when reference is made to adenine, N6-methyladenine, 8-oxo-adenine and 7-methyladenine are included; when reference is made to uracil, dihydrouracil, isouracil, N3-glycosylated uracil, pseudouracil, 5-methyluracil, N1-methylpsudouracil, 4-thiouracil and 5-hydroxymethyluracil are included; when reference is made to guanine, 1-methylguanine, 7-methylguanosine, N2,N2-dimethylguanosine, N2,N2,7-trimethylguanosine and N2,7-dimethylguanosine are included. Whenever reference is made to nucleosides or nucleotides, ribofuranose derivatives, such as 2′-deoxy, 2′-hydroxy, and 2′-O-substituted variants, such as 2′-O-methyl, are included, as well as other modifications, including 2′-4′ bridged variants. Whenever reference is made to oligonucleotides, linkages between two mononucleotides may be phosphodiester linkages as well as modifications thereof, including, phosphonoacetate, phosphodiester, phosphotriester, PS, phosphoro(di)thioate, MP, phosphoramidate linkers, phosphoryl guanidine, thiophosphoryl guanidine, sulfono phosphoramidate and the like.

The term ‘comprising’ encompasses ‘including’ as well as ‘consisting of’, e.g., a composition ‘comprising X’ may consist exclusively of X or may include something additional, e.g., X+Y. The term ‘about’ in relation to a numerical value x is optional and means, e.g., x±10%. The word ‘substantially’ does not exclude ‘completely’, e.g., a composition which is ‘substantially free from Y’ may be completely free from Y. Where relevant, the word ‘substantially’ may be omitted from the definition of the invention.

The term “complementary” as used herein refers to the fact that the AON hybridizes under physiological conditions to the target sequence. The term does not mean that each nucleotide in the AON has a perfect pairing with its opposite nucleotide in the target sequence. In other words, while an AON may be complementary to a target sequence, there may be mismatches, wobbles and/or bulges between AON and the target sequence, while under physiological conditions that AON still hybridizes to the target sequence such that the cellular RNA editing enzymes can edit the target adenosine. The term “substantially complementary” therefore also means that despite the presence of the mismatches, wobbles, and/or bulges, the AON has enough matching nucleotides between AON and target sequence that under physiological conditions the AON hybridizes to the target RNA. As shown herein, an AON may be complementary, but may also comprise one or more mismatches, wobbles and/or bulges with the target sequence, if under physiological conditions the AON is able to hybridize to its target.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand.

References to ‘hybridisation’ typically refer to specific hybridisation and exclude non-specific hybridisation. Specific hybridisation can occur under experimental conditions chosen, using techniques well known in the art, to ensure that the majority of stable interactions between probe and target are where the probe and target have at least 70%, preferably at least 80%, more preferably at least 90% sequence identity. The term ‘mismatch’ is used herein to refer to opposing nucleotides in a double stranded RNA complex which do not form perfect base pairs according to the Watson-Crick base pairing rules. In the historical sense, mismatched nucleotides are G-A, C-A, U-C, A-A, G-G, C-C, U-U pairs. In some embodiments AONs of the present invention comprise fewer than four mismatches, for example 0, 1 or 2 mismatches. Wobble base pairs are G-U, I-U, I-A, and I-C base pairs. Although a G: G pairing would be considered a mismatch, that does not necessarily mean that the interaction is unstable, which means that the term ‘mismatch’ may be somewhat outdated based on the current invention where a Hoogsteen base-pairing may be seen as a mismatch based on the origin of the nucleotide but still be relatively stable. An isolated G: G pairing in duplex RNA can for instance be quite stable, but still be defined as a mismatch.

The term ‘splice mutation’ relates to a mutation in a gene that encodes for a pre-mRNA, wherein the splicing machinery is dysfunctional in the sense that splicing of introns from exons is disturbed and due to the aberrant splicing, the subsequent translation is out of frame resulting in premature termination of the encoded protein. Often such shortened proteins are degraded rapidly and do not have any functional activity, as discussed herein. The exact mutation does not have to be the target for the RNA editing; it may be that a neighbouring or nearby adenosine in the (splice) mutation is the target nucleotide, which conversion to I fixes the splice mutation back to a normal state. The skilled person is aware of methods to determine whether normal splicing is restored, after RNA editing of the adenosine within the splice mutation site or area.

An AON according to the present invention may be chemically modified almost in its entirety, for example by providing nucleotides with a 2′-O-methylated sugar moiety (2′-O-Me), 2′-F, and/or with a 2′-O-(2-methoxy)ethyl sugar moiety (2′-MOE). However, the orphan nucleotide in an AON of the present invention is a cytidine or a cytidine analog and comprises a diF modification at the 2′ position of the sugar, and in yet a further embodiment, at least one and in another embodiment both the two neighbouring nucleotides flanking the orphan nucleotide do not comprise a 2′-O-Me modification. Complete modification wherein all nucleotides of the AON hold a 2′-O-Me modification results in a non-functional oligonucleotide as far as RNA editing goes (known in the art), presumably because it hinders the ADAR activity at the targeted position. In general, an adenosine in a target RNA can be protected from editing by providing an opposing nucleotide with a 2′-O-Me group, or by providing a guanine or adenine as opposing base, as these two nucleobases are also able to reduce editing of the opposing adenosine. Various chemistries and modification are known in the field of oligonucleotides that can be readily used in accordance with the invention. The regular internucleosidic linkages between the nucleotides may be altered by mono- or di-thioation of the phosphodiester bonds to yield phosphorothioate esters or phosphorodithioate esters, respectively. Other modifications of the internucleosidic linkages are possible, including amidation and peptide linkers. In an embodiment, the AON of the present invention comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides.

It is known in the art that RNA editing entities (such as human ADAR enzymes) edit dsRNA structures with varying specificity, depending on several factors. One important factor is the degree of complementarity of the two strands making up the dsRNA sequence. Perfect complementarity of the two strands usually causes the catalytic domain of human ADAR to deaminate adenosines in a non-discriminative manner, reacting with any adenosine it encounters. The specificity of hADAR1 and 2 can be increased by introducing chemical modifications and/or ensuring several mismatches in the dsRNA, which presumably help to position the dsRNA binding domains in a way that has not been clearly defined yet. Additionally, the deamination reaction itself can be enhanced by providing an AON that comprises a mismatch opposite the adenosine to be edited. Following the instructions in the present application, those of skill in the art will be capable of designing the complementary portion of the oligonucleotide according to their needs.

The RNA editing protein present in the cell that is of most interest to be used with AONs of the present invention is human ADAR2. It will be understood by a person having ordinary skill in the art that the extent to which the editing entities inside the cell are redirected to other target sites may be regulated by varying the affinity of the AONs according to the invention for the recognition domain of the editing molecule. The exact modification may be determined through some trial and error and/or through computational methods based on structural interactions between the AON and the recognition domain of the editing molecule. In addition, or alternatively, the degree of recruiting and redirecting the editing entity resident in the cell may be regulated by the dosing and the dosing regimen of the AON. This is something to be determined by the experimenter (in vitro) or the clinician, usually in phase I and/or II clinical trials.

The invention concerns the modification of target RNA sequences in eukaryotic, preferably metazoan, more preferably mammalian, most preferably human cells. The invention can be used with cells from any organ e.g., skin, lung, heart, kidney, liver, pancreas, gut, muscle, gland, eye, brain, blood, and the like. The invention is particularly suitable for modifying sequences in cells, tissues or organs implicated in a diseased state of a (human) subject. The cell can be located in vitro, ex vivo or in vivo. One advantage of the invention is that it can be used with cells in situ in a living organism, but it can also be used with cells in culture. In some embodiments cells are treated ex vivo and are then introduced into a living organism (e.g., re-introduced into an organism from whom they were originally derived). The invention can also be used to edit target RNA sequences in cells from a transplant or within a so-called organoid. Organoids can be thought of as three-dimensional in vitro-derived tissues but are driven using specific conditions to generate individual, isolated tissues. In a therapeutic setting they are useful because they can be derived in vitro from a patient's cells, and the organoids can then be re-introduced to the patient as autologous material which is less likely to be rejected than a normal transplant. The cell to be treated will generally have a genetic mutation. The mutation may be heterozygous or homozygous. The invention will typically be used to modify point mutations, such as N to A mutations, wherein N may be G, C, U (on the DNA level T), preferably G to A mutations, or N to C mutations, wherein N may be A, G, U (on the DNA level T), preferably U to C mutations.

Without wishing to be bound by theory, the RNA editing through hADAR2 is thought to take place on primary transcripts in the nucleus, during transcription or splicing, or in the cytoplasm, where e.g., mature mRNA, miRNA or ncRNA can be edited.

Many genetic diseases are caused by G to A mutations, and these are preferred target diseases because adenosine deamination at the mutated target adenosine will reverse the mutation to a codon giving rise to a functional, full length wild type protein, especially when it concerns PTCs.

It should be clear, that targeted editing according to the invention can be applied to any adenosine, whether it is a mutated or a wild-type nucleotide. For example, editing may be used to create RNA sequences with different properties. Such properties may be coding properties (creating proteins with different sequences or length, leading to altered protein properties or functions), or binding properties (causing inhibition or over-expression of the RNA itself or a target or binding partner; entire expression pathways may be altered by recoding miRNAs or their cognate sequences on target RNAs). Protein function or localization may be changed at will, by functional domains or recognition motifs, including but not limited to signal sequences, targeting or localization signals, recognition sites for proteolytic cleavage or co- or post-translational modification, catalytic sites of enzymes, binding sites for binding partners, signals for degradation or activation and so on. These and other forms of RNA and protein “engineering”, whether to prevent, delay or treat disease or for any other purpose, in medicine or biotechnology, as diagnostic, prophylactic, therapeutic, research tool or otherwise, are encompassed by the present invention.

The amount of AON to be administered, the dosage and the dosing regimen can vary from cell type to cell type, the disease to be treated, the target population, the mode of administration (e.g., systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change. It is possible that higher doses of AON could compete for binding to an ADAR within a cell, thereby depleting the amount of the entity, which is free to take part in RNA editing, but routine dosing trials will reveal any such effects for a given AON and a given target.

One suitable trial technique involves delivering the AON to cell lines, or a test organism and then taking biopsy samples at various time points thereafter. The sequence of the target RNA can be assessed in the biopsy sample and the proportion of cells having the modification can easily be followed. After this trial has been performed once then the knowledge can be retained, and future delivery can be performed without needing to take biopsy samples. A method of the invention can thus include a step of identifying the presence of the desired change in the cell's target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively, the change may be assessed on the level of the protein (length, glycosylation, function, or the like), or by some functional read-out, such as a (n) (inducible) current, when the protein encoded by the target RNA sequence is an ion channel, for example.

After RNA editing has occurred in a cell, the modified RNA can become diluted over time, for example due to cell division, limited half-life of the edited RNAs, etc. Thus, in practical therapeutic terms a method of the invention may involve repeated delivery of an AON until enough target RNAs have been modified to provide a tangible benefit to the patient and/or to maintain the benefits over time.

AONs of the invention are particularly suitable for therapeutic use, and so the invention provides a pharmaceutical composition comprising an AON of the invention and a pharmaceutically acceptable carrier. In some embodiments of the invention the pharmaceutically acceptable carrier can simply be a saline solution. This can usefully be isotonic or hypotonic, particularly for pulmonary delivery. The invention also provides a delivery device (e.g., syringe, inhaler, nebuliser) which includes a pharmaceutical composition of the invention.

The invention also provides an AON of the invention for use in a method for making a change in a target RNA sequence in a mammalian, preferably a human cell, as described herein. Similarly, the invention provides the use of an AON of the invention in the manufacture of a medicament for making a change in a target RNA sequence in a mammalian, preferably a human cell, as described herein.

The invention also relates to a method for the deamination of at least one specific target adenosine present in a target RNA sequence in a cell, the method comprising the steps of: providing the cell with an AON according to the invention; allowing uptake by the cell of the AON; allowing annealing of the AON to the target RNA molecule; allowing a mammalian ADAR enzyme comprising a natural dsRNA binding domain as found in the wild type enzyme to deaminate the target adenosine in the target RNA molecule to an inosine; and optionally identifying the presence of the inosine in the RNA sequence.

In a preferred aspect, depending on the ultimate deamination effect of A to I conversion, the identification step comprises: sequencing the target RNA; assessing the presence of a functional, elongated, full length and/or wild type protein; assessing whether splicing of the pre-mRNA was altered by the deamination; or using a functional read-out, wherein the target RNA after the deamination encodes a functional, full length, elongated and/or wild type protein. Because the deamination of the adenosine to an inosine may result in a protein that is no longer suffering from the mutated A at the target position, the identification of the deamination into inosine may also be a functional read-out, for instance an assessment on whether a functional protein is present, or even the assessment that a disease that is caused by the presence of the adenosine is (partly) reversed. The functional assessment for each of the diseases mentioned herein will generally be according to methods known to the skilled person. A very suitable manner to identify the presence of an inosine after deamination of the target adenosine is of course RT-PCR and sequencing, using methods that are well-known to the person skilled in the art.

The AON according to the invention is suitably administrated in aqueous solution, e.g. saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be by inhalation (e.g., through nebulization), intranasally, orally, by injection or infusion, intravenously, subcutaneously, intra-dermally, intra-cranially, intravitreally, intramuscularly, intra-tracheally, intra-peritoneally, intra-rectally, parenterally, and the like. Administration may be in solid form, in the form of a powder, a pill, a gel, an eye-drop, a solution, a slow-release formulation, or in any other form compatible with pharmaceutical use in humans.

### EXAMPLES

### Example 1. Use of 2′,2′-Difluoro Modified RNA Editing Guide Oligonucleotides for A-to-I Editing of hAPP Target RNA in an In Vitro Biochemical Editing Assay

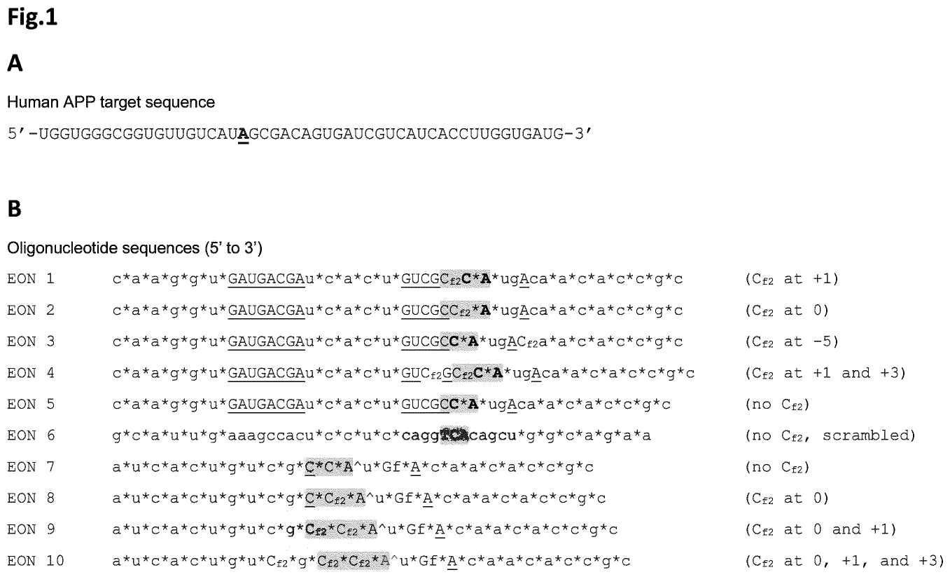

It was initially investigated whether RNA editing oligonucleotides comprising a 2′,2′-difluoro (diF) substitution in the ribose moiety of one or more nucleotides were applicable for specific editing of human Amyloid Precursor Protein (APP) target (pre-) mRNA. To study the effect of diF on hAPP target RNA editing, four initial guide oligonucleotides were designed and named EON 1, 2, 3, and 4 respectively (FIG. 1B). The editing efficacy of all four guide oligonucleotides were measured and compared to EON 5 (identical to EON 1 to 4, but without diF) and a scrambled oligonucleotide but with comparable chemical modifications in the backbone (EON 6) in an in vitro biochemical editing assay.

To obtain the hAPP target RNA a PCR was performed using a hAPP G-block (IDT) which contained the sequence for the T7 promotor and (a part of) the sequence of hAPP as template using forward primer 5′-CTC GAC GCA AGC CAT AAC AC-3′ (SEQ ID NO:20) and reverse primer 5′-TGG ACC GAC TGG AAA CGT AG-3′ (SEQ ID NO:21). The PCR product was then used as template for the in vitro transcription. The MEGAscript T7 transcription kit was used for this reaction. The RNA was purified on a urea gel then extracted in 50 mM Tris-Cl pH 7.4, 10 mM EDTA, 0.1% SDS, 0.3 M NaCl buffer and phenol-chloroform purified. The purified RNA was used as target in the biochemical editing assay.

Guide oligonucleotides EON 1 to 6 were used for annealing to the hAPP target RNA, which was done in a buffer (5 mM Tris-Cl pH 7.4, 0.5 mM EDTA and 50 mM NaCl) at the ratio 1:3 of target RNA to oligonucleotide (600 nM oligonucleotide and 200 nM target). The samples were heated at 95° C. for 3 min and then slowly cooled down to RT. Next, the editing reaction was carried out. The annealed oligonucleotide/target RNA was mixed with protease inhibitor (complete™, Mini, EDTA-free Protease I, Sigma-Aldrich), RNase inhibitor (RNasin, Promega), poly A (Qiagen), tRNA (Invitrogen) and editing reaction buffer (15 mM Tris-Cl pH 7.4, 1.5 mM EDTA, 3% glycerol, 60 mM KCl, 0.003% NP-40, 3 mM MgCl₂ and 0.5 mM DTT) such that their final concentration was 6 nM oligonucleotide and 2 nM target RNA. The reaction was started by adding purified ADAR2 (GenScript) to a final concentration of 6 nM into the mix and incubated for predetermined time points (0 sec, 30 sec, 1 min, 2 min, 5 min, 10 min, 25 min and 50 min) at 37° C. Each reaction was stopped by adding 95 μl of boiling 3 mM EDTA solution. A 6 μl aliquot of the stopped reaction mixture was then used as template for cDNA synthesis using Maxima reverse transcriptase kit with random hexamer primer (ThermoFisher Scientific). Initial denaturation of RNA was performed in the presence of the primer and dNTPs at 95° C. for 5 min, followed by slow cooling to 10° C., after which first strand synthesis was carried out according to the manufacturer's instructions in a total volume of 20 μl, using an extension temperature of 62° C. Products were amplified for pyrosequencing analysis by PCR, using the Amplitaq gold 360 DNA Polymerase kit (Applied Biosystems) according to the manufacturer's instructions, with 1 μl of the cDNA as template. The following primers were used at a concentration of 10 UM: Pyroseq Fwd hAPP, 5′-TGG GTT GAC AAA TAT CAA GAC G-3′ (SEQ ID NO:22), and Pyroseq Rev hAPP Biotin, 5′-/5BiosG/CAC CAT GAT GAA TGG ATG TGT ACT-3′ (SEQ ID NO:23). PCR was performed using the following thermal cycling protocol: Initial denaturation at 95° C. for 5 min, followed by 40 cycles of 95° C. for 30 sec, 58° C. for 30 sec and 72° C. for 30 sec, and a final extension of 72° C. for 7 min.

Because inosines base-pair with cytidines during the cDNA synthesis in the reverse transcription reaction, the nucleotides incorporated in the edited positions during PCR will be guanosines. The percentage of guanosine (edited) versus adenosine (unedited) was defined by pyrosequencing. Pyrosequencing of the PCR products and data analysis were performed by the PyroMark Q48 Autoprep instrument (QIAGEN) following the manufacturer's instructions with 10 μl input of the PCR product and 4 UM of the following sequencing primer: hAPP-Seq, 5′-GCA ATC ATT GGA CTC AT-3′ (SEQ ID NO:24). The settings specifically defined for this target RNA strand included two sets of sequence information. The first of these defines the sequence for the instrument to analyse, in which the potential for a particular position to contain either an adenosine or a guanosine is indicated by a “/”: GGT GGG CGG TGT TGT CAT A/G GCG ACA GTG ATC GTC AT (SEQ ID NO:25). The dispensation order was defined for this analysis as follows: TGT GCG TGT GTC ACT AGC GAG CAG TG (SEQ ID NO:26). The analysis performed by the instrument provides the results for the selected nucleotide as a percentage of adenosine and guanosine detected in that position, and the extent of A-to-I editing at a chosen position will therefore be measured by the percentage of guanosine in that position.

The results shown in FIG. 1C indicate that all five tested guide oligonucleotides can edit the target hAPP RNA, while EON 6 (the control scrambled oligonucleotide) did not result in any editing. Editing is highest with EON 2 and 3 that showed editing efficiencies that were comparable to what was observed with EON 5 that does not carry a diF substitution. Notable, when the diF substitution was located at position+1, immediately adjacent to the orphan nucleotide, editing efficiency was diminished (EON 1 and EON 4). This shows at least the introduction of diF substitution at position 0 (EON 2) and at position-5 (EON 3) did not influence the RNA editing efficiency significantly in the applied biochemical editing assay. As can be seen left of the diagram, the K value for EONs 1 and 2 with a diF substitution at the 2′ position at +1 and 0, respectively, is higher than observed with the control (EON 5), which may be contributing to the higher RNA editing efficiency seen with these two EONs. The K value is a measure of speed of the editing reaction; a higher K value indicates a more efficient editing reaction.

### Example 2: Cell Assay Using Endogenous ADAR and AONs Comprising Nucleotides Carrying a 2′,2′-Difluoro (diF) Modification at a Variety of Positions to Deaminate a Target Adenosine in hAPP

The same AONs as described in Example 1 and FIG. 1B were tested in immortalized human retinal pigment epithelium cells (ARPE-19) using gymnotic uptake of the AONs. For this, 1×10⁵ ARPE-19 cells were seeded in a 12 wells plate on day 0 and treated with 5 μM oligonucleotide on the same day. The next day, cells were treated with chloroquine (CQ; 40 μg/mL for 24 hrs) to enhance the uptake of the oligonucleotide. 48 hrs after adding the oligonucleotides, cells were harvested, and RNA was extracted using the Direct-zol RNA MiniPrep (Zymo Research) kit according to the manufacturer's instructions, and cDNA was prepared using the Maxima reverse transcriptase kit (Thermo Fisher) according to the manufacturer's instructions, with a combination of random hexamer and oligo-dT primers. The cDNA was diluted 5× and 1 μL of this dilution was used as template for digital droplet PCR (ddPCR). The ddPCR assay for absolute quantification of nucleic acid target sequences was performed using BioRad's QX-200 Droplet Digital PCR system. 1 μl of diluted cDNA obtained from the RT cDNA synthesis reaction was used in a total mixture of 21 μl of reaction mix, including the ddPCR Supermix for Probes no dUTP (Bio Rad), a Taqman SNP genotype assay with the following forward and reverse primers combined with the following gene-specific probes:

Forward primer:
(1205 hAPP Ex17 ddPCR Fw; SEQ ID NO: 27)
5′-CATTGGACTCATGGTGG-3′
Reverse primer:
(1206 hAPP Ex17 ddPCR Rev; SEQ ID NO: 28)
5′-CAGCATCACCAAGGTG-3′
Wild type probe (HEX NFQ labeled):
(1207 hAPP Ex17 ddPCR WT HEX; SEQ ID NO: 29)
5′-/5HEX/TGTT+GTCAT+A+G+CGACAGT/3IABkFQ/-3′
Mutant probe (FAM NFQ labeled):
(1209 hAPP Ex17 ddPCR Mut FAM_3; SEQ ID NO: 30)
5′-/56-FAM/TGTTGTCAT+G+GCGACAGT/3IABkFQ/-3′

A total volume of 21 μl PCR mix including cDNA was filled in the middle row of a ddPCR cartridge (BioRad) using a multichannel pipette. The replicates were divided by two cartridges. The bottom rows were filled with 70 μl of droplet generation oil for probes (BioRad). After the rubber gasket replacement, droplets were generated in the QX200 droplet generator. 42 μl of oil emulsion from the top row of the cartridge was transferred to a 96-wells PCR plate. The PCR plate was sealed with a tin foil for 4 sec at 170° C. using the PX1 plate sealer, followed by the following PCR program: 1 cycle of enzyme activation for 10 min at 95° C., 40 cycles denaturation for 30 sec at 95° C. and annealing/extension for 1 min at 55.8° C., 1 cycle of enzyme deactivation for 10 min at 98° C., followed by a storage at 8° C. After PCR, the plate was read and analysed with the QX200 droplet reader.

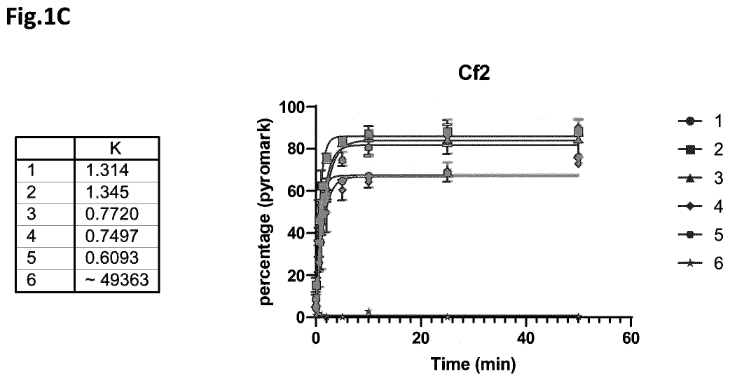

The results shown in FIG. 2 reveal that all five EONs could bring about editing of human APP target RNA in human ARPE-19 cells, and able to recruit endogenous ADAR in these cells. No editing was observed in the non-treated (NT) sample. Clearly, EON 2 with the diF substitution at position 0 (representing the orphan nucleotide) outperformed the other EONs.

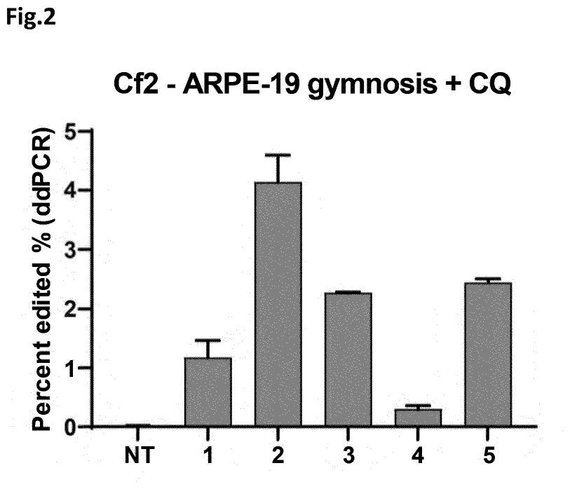

The same experiment was performed with a second set of EONs that are 25 nt in length, which is significantly shorter than the EONs that were initially tested (EONs 1 to 5 are 38 nt in length). Different to the earlier experiment was that 1.5×10⁵ cells were used per well, and that the cells were harvested for RNA purification at 72 hrs after addition of the oligonucleotides. The shorter EONs also comprised a single 2′-fluoro substitution at position-3 (see FIG. 1B) and a methylphosphonate linkage at linkage position-1 (the linkage position 3′ from the orphan nucleotide is linkage position 0). Human ARPE-19 cells were used in a transfection as well as in a gymnotic uptake experiment, generally as described above. The experiments showed the same results (transfection results not shown). FIG. 3 shows the percentage editing after gymnotic uptake of EONs 7 to 10 in comparison to non-treated (NT) cells, clearly indicating that EON 8 with a single diF substitution at position 0 outperformed the other EONs, which is in line with what was observed with the longer EONs, and especially EON 2. It appears also that shortening the oligonucleotide resulted in a higher editing percentage: approximately 4% in FIG. 2 with EON 2 and approximately 22% in FIG. 3 with EON 8, both through gymnotic uptake of the oligonucleotides.

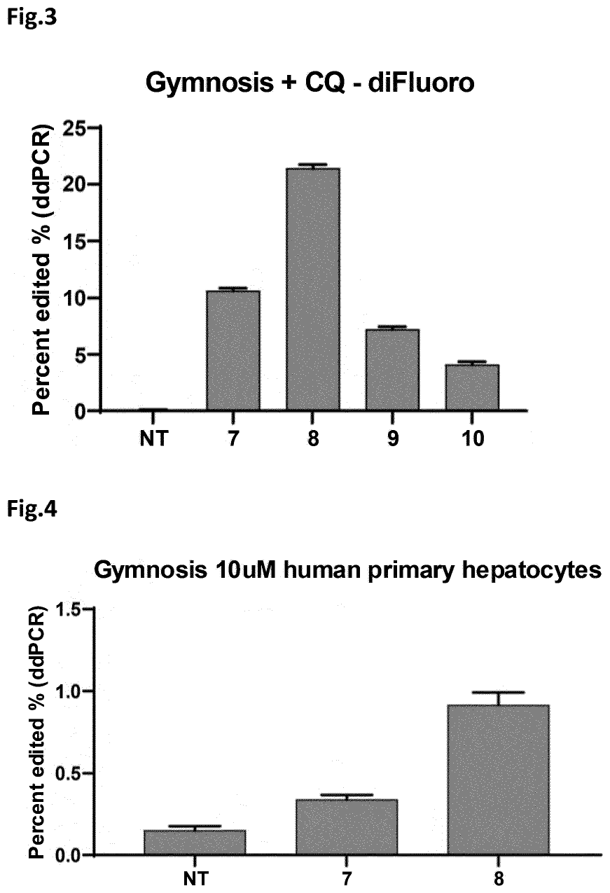

To further investigate the effect of having a diF substitution at position 0, EON 7 and EON 8 were tested for bringing about RNA editing in primary human hepatocytes. For this, 1×10⁵ primary hepatocytes were seeded per well in a 96-well plate that was coated with collagen I. 16 hrs post seeding cells, 10 UM oligonucleotide was added for gymnotic uptake and cells were harvested 48 hrs after oligonucleotide treatment. FIG. 4 shows that the editing percentage in these human primary hepatocytes is significantly lower than observed with human ARPE-19 cells, but the results also show that EON 8 performed at least twice as good as EON 7. It should be noted that the only difference between these oligonucleotides is the diF substitution at the orphan cytidine, clearly indicating the beneficial properties of having this modification at that position.

### Example 3: Cell Assay Using Endogenous ADAR and AONs Comprising Nucleotides Carrying a 2′,2′-Difluoro (diF) Modification at the Orphan Nucleotide to Deaminate a Target Adenosine in mAPP

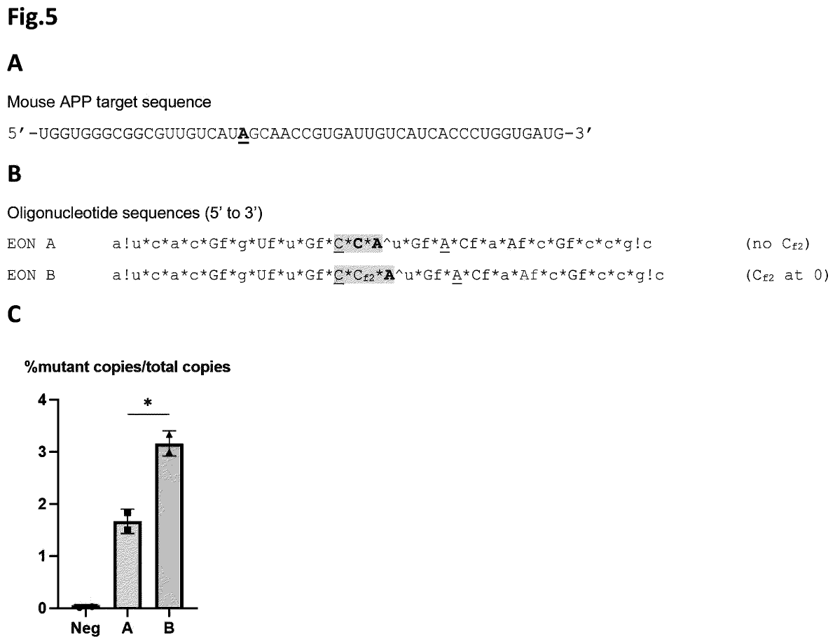

In a next experiment, two oligonucleotides, with additional modifications were tested in mouse cells, hence with a mouse target APP RNA sequence. The two oligonucleotides (EON A and EON B) with their specific chemical modifications are shown in FIG. 5B, while the target sequence is shown in FIG. 5A. For this, mouse retinal pigment epithelium (RPE) cells carrying the wild-type APP gene were used. Briefly, 2.5×10⁵ cells per 6 well plate were seeded 24 h before oligonucleotide treatment, which was done with 5 μM EON, while a non-treated control sample did not receive oligonucleotide. RNA was harvested and a ddPCR assay was performed as described in Example 2.

FIG. 5C shows the results, again clearly indicating that the oligonucleotide which carried a diF substitution at position 0 (EON B) outperformed the oligonucleotide that did not carry a diF substitution anywhere in the molecule (EON A). These experiments show the beneficial properties of oligonucleotides that can be used for RNA editing of a single adenosine in a target RNA molecule in a cell, using endogenous deaminating enzymes (such as ADAR2), wherein the oligonucleotide comprises a 2′,2′-difluoro modification at the orphan nucleotide opposite the target adenosine.

### Example 4: RNA Editing of Human PCSK9 Target RNA Using AONs Carrying a 2′,2′-Difluoro (diF) Modification at the Orphan Nucleotide

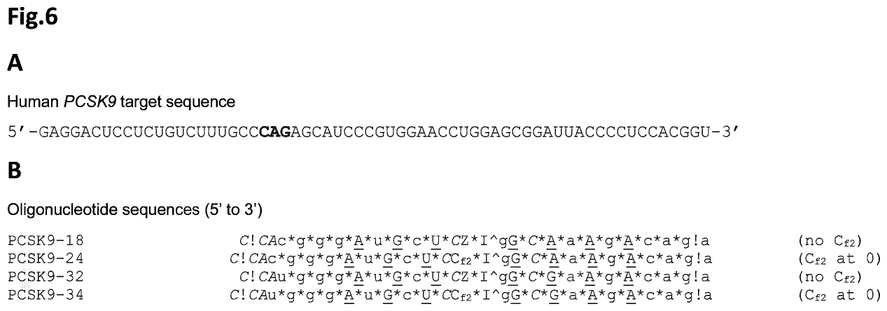

The PCSK9 enzyme is central in the regulation of the LDL receptor, a receptor involved in the uptake of Low-Density Lipoprotein Cholesterol (LDL-C; the “bad” cholesterol) from the blood. PCSK9 is predominantly synthesized in the liver in hepatocytes. In patients with hypercholesterolemia, familial or otherwise, wherein high levels of plasma circulating LDL-C is present, the LDL receptor is downregulated and/or degraded upon interaction with the PCSK9 protein, a process that requires processing of the PCSK9 proprotein by autocleavage at the P1 site (Benjannet S et al. J Biol Chem. 2012 287 (40): 33745-33755). The target adenosine for editing as shown herein is the second nucleotide of the codon coding for the glutamine residue at position 152 in the human PCSK9 proprotein. This position is often also referred to as Gln152. The Gln152 residue is part of the P1 cleavage site of the PCSK9 proprotein. The applied antisense RNA editing oligonucleotides as used herein are substantially complementary to a region within the human PCSK9 pre-mRNA or mRNA that comprises the codon coding for the glutamine residue at position 152 in the PCSK9 proprotein. Here, four oligonucleotides were used, two (referred to as PCSK9-18 and PCSK9-24) being 100% complementary to the human PCSK9 sequence (except for the mismatch between the orphan nucleotide in the oligonucleotide and the target adenosine) and two (referred to as PCSK9-32 and PCSK9-34) being 100% complementary to the monkey PCSK9 sequence (also except for the mismatch between the orphan nucleotide in the oligonucleotide and the target adenosine). The orphan nucleotide in PCSK9-18 and PCSK9-32 is a deoxynucleotide (DNA) carrying a Benner's base and is abbreviated in the sequence to Z in FIG. 6B. The orphan nucleotide in PCSK9-24 and PCSK9-34 is a cytidine carrying a 2′,2′-difluoro modification in the sugar moiety and is abbreviated to C_{f2} in FIG. 6B. PCT/EP2023/053503 (not published), which is herein incorporated in its entirety, discloses the rationale and further details related to PCSK9 editing. The example shown here is to provide a further insight in the use of the 2′,2′-difluoro modification at the orphan nucleotide.

A set of four oligonucleotides was tested for A to I editing of a wild type PCSK9 target RNA in human cervix carcinoma cells (HeLa). FIG. 6 shows part of the (pre) mRNA target sequence of the human PCSK9 transcription product. PCSK9-18, -24, -32, and -34 were tested for producing RNA editing in the HeLa cells after transfection, using a scrambled oligonucleotide and mock transfection as negative controls.

Cell culture: Human Hela cells were cultured in DMEM/10% FBS and Human HepG2 hepatocellular carcinoma cells were cultured in MEM/10% FBS. Cells were kept at 37° C. in a 5% CO₂ atmosphere. Primary human hepatocytes were provided by Primacyt and cultured in serum-free Human Hepatocyte Maintenance Medium (Primacyt) and kept at 37° C. in a 10% CO₂ atmosphere.

Transfection: A total of 0.5×10⁵ Hela cells were transfected using Lipofectamine 2000 (Invitrogen) according to the manufacturer's instructions. In brief, oligonucleotides were diluted in OptiMem (Thermo Fisher) to a final concentration of 100 nM and mixed by vortexing 1:1 with a mixture of Lipofectamine 2000 in OptiMem in a 1:4.4 ratio (oligonucleotide: transfection agent). After incubation at RT for 20 min, mixtures were added dropwise to the wells containing fresh medium. After 24 hrs, the inoculate was aspirated and fresh culture medium was added to the wells.

RNA isolation: 48 hrs post initial exposure to the oligonucleotides, cells were collected, and total RNA was isolated from the transfected cells using the Direct-zol RNA Microprep kit (Zymo Research). After removal of the culture medium, cells were washed once with PBS. After complete aspiration of the PBS, 100 UL TRIreagent (Zymo Research) was added to lyse the cells and collect the intracellular material. After addition of 100 UL ethanol, the mixtures were loaded in a column and subjected to several wash steps and DNasel treatment. After elution in a total volume of 15 μL DNase/RNase-free water, the RNA yield was determined using spectrophotometric analysis (NanoDrop) and stored at −80° C.

Generation of cDNA: Maxima reverse transcriptase (RT, Thermo Fisher) was used to generate cDNA. Typically, 200 ng total RNA was used in reaction mixture containing 4 μL 5×RT buffer, 1 μL dNTP mix (10 mM each), 0.5 μL Oligo (dT), 0.5 μL random hexamer (all Thermo Fisher) supplemented with DNase and RNase free water to a total volume of 20 μL. Samples were loaded in a T100 thermocycler (Bio-Rad) and initially incubated at 10 min at 25° C., followed by a cDNA reaction temperature of 30 min at 50° C. and a termination step of 5 min at 85° C. Samples were cooled down to 4° C. prior storing at −20° C.

Digital droplet PCR (ddPCR) to determine RNA editing: cDNA samples were used in two multiplex ddPCR assays. The first ddPCR is designed to distinguish between cDNA species containing the original adenosine or the edited inosine, which is converted into a guanidine during cDNA synthesis. The second multiplex ddPCR quantifies the amount of PCSK9 specific cDNA molecules in the mixture using a primer/probe set targeting exons 1 and 4 or exons 9 and 10. The primer and probe sequences are listed in the table below.

Primer and probe names and sequences (+ refers to a LNA nucleotide at the 3′ side)

| Name | Sequence 5′ to 3′ |
|---|---|
| PCSK9 ex2-3 Fw | TGCTGGAGCTGGCCTTGAAGTTG (SEQ ID NO: 31) |
| PCSK9 ex3 Rv | CTGGTATTCATCCGCCCGGTAC (SEQ ID NO: 32) |
| PCSK9 ex1 Fw | TTCCGAGGAGGACGGC (SEQ ID NO: 33) |
| PCSK9 ex4 Rv | GTCTAGGAGATACACCTCCACC (SEQ ID NO: 34) |
| PCSK9 ex9 Fw | AGTTTCTCCAGGAGTGGGAAG (SEQ ID NO: 35) |
| PCSK9 ex10 Rv | AGCACCTGGCAATGGCGTAG (SEQ ID NO: 36) |
| PCSK9 A probe | /5HEX/CTTTGCC+C+A+GAGCATCC/3IABkFQ/ (SEQ ID NO: 37) |
| PCSK9 G probe | /56-FAM/CTTTGCC+C+G+GAGCATCC/3IABkFQ/ (SEQ ID NO: 38) |
| PCSK9 ex9-10 probe | /5HEX/AGCGCATGGAGGCCCAAG/3IABkFQ/ (SEQ ID NO: 39) |
| PCSK9 ex3 probe | /56-FAM/CTGCTGGAGCTGACGGAGGC/3IABkFQ/ (SEQ ID NO: 40) |

The cycling conditions were as follows: enzyme activation at 95° C. for 10 min, then 40 cycles of denaturation at 95° C. for 30 sec, annealing/extension at 64° C. for 1 min, followed by enzyme deactivation at 98° C. for 10 min, after which the product was stored at 4° C.

In total, 0.5 μL of the cDNA mix was used in a ddPCR mixture containing 10.5 μL 2×ddPCR Supermix for probes (Bio-Rad), 1.3 μL of primers and probes (10 UM stock concentration), supplemented with 4.8 μL DNase and RNase free water in a total volume of 21 μL. Droplets were generated using the droplet generator (Bio-Rad) with the aid of 70 μL droplet generation oil (Bio-Rad). The resulting 40 μL of droplets were transferred to a 96-well plate (Eppendorf), sealed with a tin foil using a PX1 plate sealer (Bio-Rad) before loading the plate into a T100 thermal cycler. After the cycling steps, the plate was transferred to a QX200 droplet reader set to measure the Fam and Hex fluorophores. Data was analyzed using the QuantaSoft Analysis Pro software (Bio-Rad). Percentage of A-to-I editing was determined by dividing the number of G-containing molecules by the total (G-plus A-containing species) multiplied by 100.

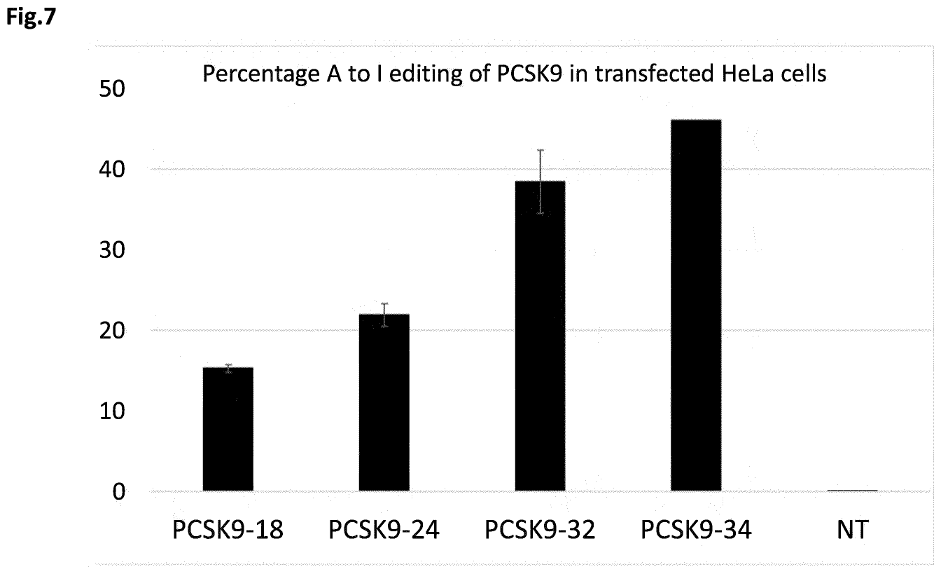

The results after transfection in Hela cells are given in FIG. 7 and clearly shows the beneficial properties of the editing oligonucleotides carrying a 2′,2′-difluoro (diF) modification in the sugar over oligonucleotides carrying a deoxynucleotide with a Benner's base (Z) at the orphan site. Interestingly, oligonucleotides PCSK9-32 and PCSK9-34 performed best in these human cells, whereas they were designed to be 100% complementary to the monkey sequence. As shown earlier in for instance WO2017/220751 this may be due to the higher efficiency in which ADAR can bind to the dsRNA target when there are certain mismatches between the target strand and the strand that forms the double-stranded complex.

Subsequently, the oligonucleotides given in FIG. 6B were used for gymnotic uptake (=no transfection, just exposure) in human hepatocytes to determine whether the oligonucleotides could produce RNA editing of an endogenous PCSK9 target RNA in liver cells. For the gymnotic treatment of the human primary hepatocytes, 1.0×10⁵ cells were seeded in a collage-coated 96-well plate. The following day, 100 μL of fresh medium containing 1, 3 or 10 UM of each oligonucleotide was added to the cells. After 48 hrs incubation, the medium was aspirated, and total RNA was isolated using the experimental set-up described above. Determination of RNA editing was performed generally as described above.

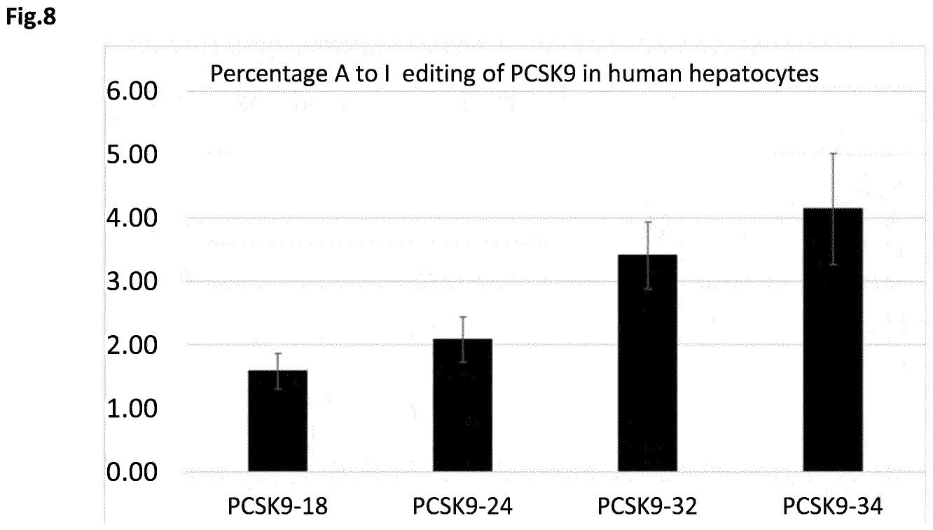

The results of this gymnotic uptake experiment with PCSK9-18, -24, -32, and -34 are shown in FIG. 8, again indicating that oligonucleotides PCSK9-32 and PCSK9-34 performed best. These results further substantiate the finding that a 2′,2′-difluoro (diF) modification at the orphan nucleotide is a beneficial chemical modification that allows very sufficient deamination of a target adenosine in a target sequence using endogenous available editing enzymes, such as ADAR.

1. An antisense oligonucleotide (AON) that can form a double stranded nucleic acid complex with a target RNA molecule, wherein the double stranded nucleic acid complex is capable of recruiting an adenosine deaminating enzyme for deamination of a target adenosine in the target RNA molecule, wherein the nucleotide in the AON that is opposite the target adenosine is the orphan nucleotide, and wherein the orphan nucleotide has the structure of formula I: 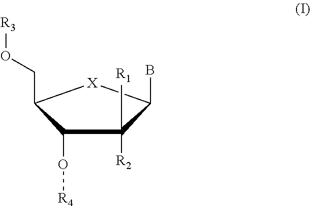
wherein:
X is O, NH, CH₂, Se, or S;
B is a nitrogenous base selected from the group consisting of: cytosine, uracil, isouracil, N3-glycosylated uracil, pseudoisocytosine, 8-oxo-adenine, and 6-amino-5-nitro-2(1H)-pyridone;
R₁ and R₂ are both selected, independently, from OH, F or CH₃;
R₃ is the part of the AON that is 5′ of the orphan nucleotide, consisting of 7 to 30 nucleotides; and
R₄ is the part of the AON that is 3′ of the orphan nucleotide, consisting of 4 to 25 nucleotides.

2. The AON according to claim 1, wherein R₁ or R₂ is F, and wherein R₁ or R₂ is either CH₃ or also F.

3. The AON according to claim 1, wherein R₁ and R₂ are F.

4. The AON according to any one of claims 1 to 3, wherein one or more nucleotides in R₃ and/or R₄ comprise a chemical modification that is a mono- or di-substitution at the 2′,3′ and/or 5′ position of the ribose sugar, selected from the group consisting of:
—OH;
—F;
substituted or unsubstituted, linear or branched lower (C₁-C₁₀) alkyl, alkenyl, alkynyl, alkaryl, allyl, or aralkyl, that may be interrupted by one or more heteroatoms;
—O—, S—, or N-alkyl;
—O—, S—, or N-alkenyl;
—O—, S—, or N-alkynyl;
—O—, S—, or N-allyl;
—O-alkyl-O-alkyl;
-methoxy;
-aminopropoxy;
-methoxyethoxy;
-dimethylamino oxyethoxy; and
-dimethylaminoethoxyethoxy.

5. The AON according to any one of claims 1 to 4, wherein the AON comprises at least one phosphorothioate, phosphonoacetate, phosphorodithioate, methylphosphonate, sulfonylphosphoramidate, or PNdmi internucleotide linkage.

6. The AON according to any one of claims 1 to 5, wherein the AON consists of 15, 16, 17, 18, 19, 20, 21, 22, 23, 24, 25, 26, 27, 28, 29, 30, 31, 32, 33, 34, 35, 36, 37 or 38 nucleotides.

7. The AON according to any one of claims 1 to 6, wherein the adenosine deaminating enzyme is an endogenous ADAR enzyme, preferably ADAR2.

8. A pharmaceutical composition comprising an AON according to any one of claims 1 to 7, and a pharmaceutically acceptable carrier or diluent.

9. An AON according to any one of claims 1 to 7, or a pharmaceutical composition according to claim 8, for use in the treatment, amelioration, slowing down progression, or prevention of a genetic disease caused by a premature termination codon.

10. A method for the deamination of at least one target adenosine present in a target RNA molecule in a cell, the method comprising the steps of:
(i) providing the cell with an AON according to any one of claims 1 to 7, or a pharmaceutical composition according to claim 8;
(ii) allowing annealing of the AON to the target RNA molecule to form a double stranded nucleic acid complex capable of recruiting an adenosine deaminating enzyme in the cell, preferably an endogenous adenosine deaminating enzyme, more preferably ADAR2;
(iii) allowing the adenosine deaminating enzyme to deaminate the target adenosine in the target RNA molecule; and
(iv) optionally identifying the presence of the deaminated adenosine in the target RNA molecule.

11. The method of claim 10, wherein step (iv) comprises:
a) sequencing a region of the target RNA molecule, wherein the region comprises the deaminated target adenosine;
b) assessing the presence of a functional, elongated, full length and/or wild type protein when the target adenosine is in a stop codon; or
c) using a functional read-out, wherein the target RNA molecule after the deamination encodes a functional, full length, elongated and/or wild type protein.

12. A method for the deamination of at least one target adenosine present in a target RNA molecule, the method comprising the steps of:
(i) providing an AON according to any one of claims 1 to 7;
(ii) allowing annealing of the AON to the target RNA molecule to form a double stranded nucleic acid complex with the target RNA molecule;
(iii) allowing a mammalian adenosine deaminating enzyme to deaminate the target adenosine in the target RNA molecule; and
(iv) optionally identifying the presence of the deaminated adenosine in the target RNA molecule.

13. A method according to claim 12, wherein the adenosine deaminating enzyme is an endogenous ADAR enzyme, preferably ADAR2.

14. An AON according to any one of claims 1 to 7, or a pharmaceutical composition according to claim 8, for use in the treatment or prevention of a genetic disorder, preferably selected from the group consisting of: Hurler Syndrome, alpha-1-antitrypsin (A1AT) deficiency, (familial) hypercholesterolemia, Parkinson's disease, Rett syndrome, Stargardt Disease, Citrullinemia Type 1, autosomal recessive non-syndromic hearing loss, X-linked retinoschisis, argininosuccinate lyase deficiency, Duchenne/Becker muscular dystrophy, Non-Alcoholic Steatohepatitis (NASH), Myotonic dystrophy type I, Myotonic dystrophy type II, Huntington's disease, Usher syndrome (such as Usher syndrome type I, II, and III), Charcot-Marie-Tooth disease, Cystic fibrosis, Alzheimer's disease, albinism, Amyotrophic lateral sclerosis, Asthma, β-thalassemia, Epileptic Encephalopathy, CADASIL syndrome, Chronic Obstructive Pulmonary Disease (COPD), Distal Spinal Muscular Atrophy (DSMA), Dystrophic Epidermolysis bullosa, Epidermolysis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hereditary Cancer predisposing Syndrome, Hunter Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, neurofibromatosis, Niemann-Pick disease type A, B and C, NY-eso1 related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, (autosomal dominant) Retinitis Pigmentosa, Sandhoff Disease, Severe Combined Immune Deficiency Syndrome (SCID), Sickle Cell Anaemia, Spinal Muscular Atrophy, Tay-Sachs Disease, X-linked immunodeficiency, Sturge-Weber Syndrome, and cancer, such as breast and lung cancer.
